(19) United States
(12) Patent Application Publication
(10) Pub. No.: US 2011/0091994 A1
(43) Pub. Date: Apr. 21, 2011

(54) Methods for Screening Compounds for Treating and/or Preventing an Hepatitis C Virus Infection

(75) Inventor: Vincent Lotteau, Vourles (FR)

(73) Assignee: Inserm (Institut National de la Santa et de la Recherche Medicale)

(21) Appl. No.: 12/934,144

(22) PCT Filed: Apr. 16, 2009
(86) PCT No.: PCT/EP09/54536

§ 371 Date: Dec. 9, 2010

(30) Foreign Application Priority Data
Apr. 16, 2008 (IB) PCT/IB08/53132

(51) Int. Cl. G01N 33/53

(52) U.S. Cl. 436/501

(57) ABSTRACT

The present invention relates to methods for screening compounds for treating and/or preventing an Hepatitis C Virus (HCV) infection.

### FIELD OF THE INVENTION

The present invention relates to methods for screening compounds for treating and/or preventing an Hepatitis C Virus (HCV) infection.

### BACKGROUND OF THE INVENTION

Hepatitis C Virus (HCV) infection is characterized by a high rate of chronicity and concerns 170 millions of individuals worldwide. Chronically-infected patients present liver injury essentially mediated by immune mechanisms and metabolic disorders associated with hepatic steatosis, fibrogenesis and insulin resistance to various extent (1, 2). Long-term infected patients have a high risk of developing cirrhosis and hepatocarcinoma but despite considerable efforts, molecular basis of HCV pathology remains poorly understood. HCV genome is a positive strand RNA of 9.6 kb encoding a polyprotein that is post-translationally processed into structural (CORE, E1, E2 and p7) and non structural (NS2, NS3, NS4A, NS4B, NS5A and NS5B) proteins (3).

Current therapy consists in the association of pegylated interferon (IFN) alpha and ribavirin (1-β-D-ribofuranosyl-1,2,4-triazole-3-carboxamide). However, the outcome of hepatitis C virus (HCV) infection varies among individuals and the likelihood of sustained response to antiviral treatment depends on viral and host characteristics. Naturally occurring variants of HCV are classified into 6 major genotypes. Viral genotype is one of the main factors associated to therapy response. Indeed, sustained virological response (SVR) is achieved in only 45% of the genotype 1 infected patients, whereas up to 80% of the genotypes 2 or 3 infected patients reach a SVR (Feld J J. et al. 2005).

Therefore, there is a need for other treatments of HCV infections, and there is an incentive to focus on the interactions between HCV proteins and host (human) proteins. The rapidly growing knowledge of cellular protein network and now of viral-cellular interactome indeed begins to provide network-based models for disease. In a network approach, a viral infection can be viewed as a perturbation of the cellular interactome. Viral pathogenesis appears as the expression of new constraints on the protein network imposed by the virus when connecting to the cellular interactome. Identification of topological and functional properties that are lost, dysregulated or that emerge in the “infected network” becomes a major challenge for the complex systems analysis of an infection. However, the interactions between human and viral proteins have not yet fully documented.

### SUMMARY OF THE INVENTION

The present invention relates to a method for screening compounds for treating and/or preventing an HCV infection comprising the steps of:
a) determining the ability of a candidate compound to inhibit the interaction between the farnesoid X receptor (FXR) and viral HCV protein NS3 or NS5A;
b) selecting the candidate compound that inhibits said interaction between said viral farnesoid X receptor (FXR) and said viral protein.

The farnesoid X receptor (FXR) is a nuclear receptor that is activated by supraphysiological levels of farnesol (Forman et al., Cell, 1995, 81, 687-693). FXR, is also known as NR1H4, retinoid X receptor-interacting protein 14 (RIP14) and bile acid receptor (BAR).

The present invention also relates to a method for screening compounds for treating and/or preventing an HCV infection comprising the steps of:
a) determining the ability of a candidate compound to inhibit the interaction between the viral HCV CORE protein and a human protein selected from the group consisting of AGRN, BCAR1, CD68, COL4A2, DDX3Y, EGFL7, FBLN2, FBLN5, GAPDH, GRN, HIVEP2, HOXD8, LPXN, LRRTM1, LTBP4, MAGED1, MEGF6, MMRN2, NR4A1, PABPN1, PAK4, PLSCR1, RNF31, SETD2, SLC31A2, VTN, VWF, and ZNF271; and
b) selecting the candidate compound that inhibits said interaction between said viral HCV CORE protein and said human protein.

The present invention also relates to a method for screening compounds for treating and/or preventing an HCV infection comprising the steps of:
a) determining the ability of a candidate compound to inhibit the interaction between the viral HCV E1 protein and a human protein selected from the group consisting of JUN, NR4A1, PFN1, SETD2, and TMSB4X; and
b) selecting the candidate compound that inhibits said interaction between said viral E1 protein and said human protein.

The present invention also relates to a method for screening compounds for treating and/or preventing an HCV infection comprising the steps of:
a) determining the ability of a candidate compound to inhibit the interaction between the viral HCV E2 protein and a human protein selected from the group consisting of HOXD8, ITGB1, KIAA1411, LOC730765, NR4A1, PSMA6, SETD2, and SMEK2; and
b) selecting the candidate compound that inhibits said interaction between said viral E2 protein and said human protein.

The present invention also relates to a method for screening compounds for treating and/or preventing an HCV infection comprising the steps of:
a) determining the ability of a candidate compound to inhibit the interaction between the viral HCV NS2 protein and a human protein selected from the group consisting of ADFP, APOA1, C7, FBLN5, HOXD8, NR4A1, POU3F2, RPL11, RPN1, SETD2, SMURF2, and TRIM27; and
b) selecting the candidate compound that inhibits said interaction between said viral NS2 protein and said human protein.

The present invention also relates to a method for screening compounds for treating and/or preventing an HCV infection comprising the steps of:
a) determining the ability of a candidate compound to inhibit the interaction between the viral HCV NS3 protein and a human protein selected from the group consisting of sep-10, A1BG, ABCC3, ACTN1, ACTN2, AEBP1, AHCY, AHSG, ALB, ANKRD12, ANKRD28, APOA1, APOA2, ARFIP2, ARG1, ARHGDIA, ARHGEF6, ARNT, ARS2, ASXL1, ATP5H, AZGP1, B2M, BCAN, BCKDK, BCL2A1, BCL6, BCR, BZRAP1, C10orf18, C10orf6, C12orf41, C14orf173, C16orf7, C1orf165, C1orf94, C1S, C9orf30, CALCOCO2, CAT, CBY1, CCDC21, CCDC37, CCDC52, CCDC66, CCDC95, CCHCR1, CCNDBP1, CD5L, CDC23, CELSR2, CENPC1, CEP152, CEP192, CES1, CFP, CHPF, COL3A1, CORO1B, COX3, CSNK2B, CTGF, CTSD, CTSF, CXorf45, DEAF1, DEDD2, DES, DLAT, DOCK7, DPF1, DPP7, ECHS1, EEF1A1, EFEMP1, EFEMP2, EIF1, EIF4ENIF1, FAM120B, FAM62B, FAM65A, FAM96B, FBF1, FBLN1, FBLN2, FBLN5, FBN1, FBN3, FES, FGA, FGB, FIGNL1, FLAD1, FLJ11286, FN1, FRMPD4, FRS3, FTH1, FUCA2, GAA, GBP2, GC, GFAP, GNB2, GON4L, HIVEP2, HOMER3, HP, HTRA1, IFI44, IQWD1, ITCH, ITGB4, JAG2, JUN, KHDRBS1, KIAA1012, KIAA1549, KIF17, KIF7, KNG1, KPNA1, KPNB1, L3 MBTL3, LAMA5, LAMB2, LAMC3, LDB1, LOC728302, LRRC7, LRRCC1, LTBP4, LZTS2, MAGED1, MAPK7, MARCO, MASP2, MEGF8, MLLT4, MLXIP, MORC4, MORF4L1, MPDZ, MVP, MYL6, NAP1L1, NCAN, NDC80, NEFL, NEFM, NELL1, NELL2, NID1, NID2, NOTCH1, N-PAC, NUCB1, NUP133, NUP62, OBSCN, ORM1, OTC, PARP2, PARP4, PCYT2, PDE4DIP, PDLIM5, PGM1, PICK1, PKNOX1, PLEKHG4, PNPLA8, PNPT1, POLDIP2, PRG4, PRRC1, PSMA6, PSMB9, PSME3, PTPRF, PTPRN2, RABEP1, RAI14, RASAL2, RBM4, RCN3, RGNEF, RICS, RING1, RINT1, RLF, RNF31, ROGDI, RP11-130N24.1, RSHL2, RUSC2, SBF1, SDCCAG8, SECISBP2, SELO, SERTAD1, SESTD1, SF3B2, SGCB, SIAH1, SLIT1, SLIT2, SLIT3, SMARCE1, SMURF2, SNX4, SPOCK3, SPON1, SPP2, SRPX2, SSX2IP, STAB1, STAT3, STRAD, SVEP1, SYNE1, SYNPO2, TAF1, TAF15, TBC1D2B, TBN, TBXAS1, TF, TGFB1I1, TH1L, THAP1, TMEM63B, TPST2, TPT1, TRIM23, TRIM27, TRIO, TRIP11, TXNDC11, UBE1C, USHBP1, UXT, VCAN, VIM, VWF, WDTC1, XAB2, XRN2, YY1AP1, ZADH1, ZBTB1, ZCCHC7, ZHX3, ZMYM2, ZNF281, ZNF410, ZNF440, ZXDC, and ZZZ3, APOA1, and DNAJB1; and
b) selecting the candidate compound that inhibits said interaction between said viral NS3 protein and said human protein.

The present invention also relates to a method for screening compounds for treating and/or preventing an HCV infection comprising the steps of:
a) determining the ability of a candidate compound to inhibit the interaction between the viral HCV NS4A protein and a human protein selected from the group consisting of CREB3, ELAC2, HOXD8, NR4A1, TRAF3IP3, UBQLN1, APOA1, and DNAJB1; and
b) selecting the candidate compound that inhibits said interaction between said viral NS4A protein and said human protein.

The present invention also relates to a method for screening compounds for treating and/or preventing an HCV infection comprising the steps of:
a) determining the ability of a candidate compound to inhibit the interaction between the viral HCV NS4B protein and a human protein selected from the group consisting of APOA1, ATF6, KNG1, and NR4A1; and
b) selecting the candidate compound that inhibits said interaction between said viral NS4B protein and said human protein.

The present invention also relates to a method for screening compounds for treating and/or preventing an HCV infection comprising the steps of:
a) determining the ability of a candidate compound to inhibit the interaction between the viral HCV NS5A protein and a human protein selected from the group consisting of AARS2, ABCC3, ACLY, ACTB, ALDOB, APOB, ARFIP1, ASXL1, AXIN1, C10orf30, C9orf23, CADPS, CADPS2, CCDC100, CCDC90A, CCT7, CEP250, CEP63, CES1, CFH, COL3A1, DDX5, DNAJA3, EFEMP1, EIF3S2, ETFA, FGB, FHL2, GLTSCR2, GOLGA2, GPS2, HRSP12, IGLL1, ITGAL, LDHD, LIMS2, LOC374395, MAF, MBD4, MKRN2, MOBK1B, MON2, NAP1L1, NFE2, NUCB1, OS9, PARVG, PMVK, POMP, PPP1R13L, PSMB8, PSMB9, RLF, RPL18A, RRBP1, SHARPIN, SMYD3, SORBS2, SORBS3, THBS1, TMF1, TP53BP2, TRIOBP, TST, TXNDC11, UBASH3A, UBC, USP19, VPS52, ZGPAT, ZH2C2, ZNF135, ZNF350, ZNF646, ZNHIT1, and ZNHIT4; and
b) selecting the candidate compound that inhibits said interaction between said viral NS5A protein and said human protein.

The present invention also relates to a method for screening compounds for treating and/or preventing an HCV infection comprising the steps of:
a) determining the ability of a candidate compound to inhibit the interaction between the viral HCV NS5B protein and a human protein selected from the group consisting of APOA1, APOC3, CCNDBP1, CEP250, CEP68, CTSF, HOXD8, MGC2752, MOBK1B, OS9, OTC, PKM2, PSMB9, SETD2, SHARPIN, TAGLN and TUBB2C; and
b) selecting the candidate compound that inhibits said interaction between said viral NS5B protein and said human protein.

The present invention also relates to a method for screening compounds for treating and/or preventing an HCV infection comprising the steps of:
a) determining the ability of a candidate compound to inhibit the interaction between the viral HCV p7 protein and a human protein selected from the group consisting of CREB3, FBLN2, FXYD6, LMNB1, RNUXA, SLC39A8, SLIT2, UBQLN1, and UBQLN4; and
b) selecting the candidate compound that inhibits said interaction between said viral HCV p7 protein and said human protein.

### DETAILED DESCRIPTION OF THE INVENTION

### Definitions

The term “hepatitis C virus” or “HCV” is used herein to define a viral species of which pathogenic strains cause hepatitis C, also known as non-A, non-B hepatitis. All the human and HCV genes and proteins are defined in the table 1:

TABLE 1

| | | | | Interaction |
|---|---|---|---|---|
| | | | Gene | with viral |
| Origin | Symbol | Full Name | ID | protein |
| Human | AGRN | XP_001126326 | 375790 | CORE |
| Human | BCAR1 | NP_055382 | 9564 | CORE |
| Human | CD68 | NP_001242 | 968 | CORE |
| Human | COL4A2 | NP_001837 | 1284 | CORE |
| Human | DDX3Y | NP_004651 | 8653 | CORE |
| Human | EGFL7 | NP_057299 | 51162 | CORE |
| Human | FBLN2 | NP_001004019 | 2199 | CORE |
| Human | FBLN5 | NP_006320 | 10516 | CORE |
| Human | GAPDH | NP_002037 | 2597 | CORE |
| Human | GRN | NP_002078 | 2896 | CORE |
| Human | HIVEP2 | NP_006725 | 3097 | CORE |
| Human | HOXD8 | NP_062458 | 3234 | CORE |
| Human | LPXN | NP_004802 | 9404 | CORE |
| Human | LRRTM1 | NP_849161 | 347730 | CORE |
| Human | LTBP4 | NP_003564 | 8425 | CORE |
| Human | MAGED1 | NP_008917 | 9500 | CORE |
| Human | MEGF6 | NP_001400 | 1953 | CORE |
| Human | MMRN2 | NP_079032 | 79812 | CORE |
| Human | NR4A1 | NP_002126 | 3164 | CORE |
| Human | PABPN1 | NP_004634 | 8106 | CORE |
| Human | PAK4 | NP_005875 | 10298 | CORE |
| Human | PLSCR1 | NP_066928 | 5359 | CORE |
| Human | RNF31 | NP_060469 | 55072 | CORE |
| Human | SETD2 | NP_054878 | 29072 | CORE |
| Human | SLC31A2 | NP_001851 | 1318 | CORE |
| Human | VTN | ENSP00000226218 | | CORE |
| Human | VWF | NP_000543 | 7450 | CORE |
| Human | ZNF271 | NP_006620 | 10778 | CORE |
| Human | JUN | NP_002219 | 3725 | E1 |
| Human | NR4A1 | NP_002126 | 3164 | E1 |
| Human | PFN1 | NP_005013 | 5216 | E1 |
| Human | SETD2 | NP_054878 | 29072 | E1 |
| Human | TMSB4X | NP_066932 | 7114 | E1 |
| Human | HOXD8 | NP_062458 | 3234 | E2 |
| Human | ITGB1 | NP_002202 | 3688 | E2 |
| Human | KIAA1411 | NP_065870 | 57579 | E2 |
| Human | LOC730765 | XP_001127129 | 730765 | E2 |
| Human | NR4A1 | NP_002126 | 3164 | E2 |
| Human | PSMA6 | NP_002782 | 5687 | E2 |
| Human | SETD2 | NP_054878 | 29072 | E2 |
| Human | SMEK2 | NP_065196 | 57223 | E2 |
| Human | ADFP | ENSP00000369832 | | NS2 |
| Human | APOA1 | ENSP00000236850 | | NS2 |
| Human | C7 | NP_000578 | 730 | NS2 |
| Human | FBLN5 | NP_006320 | 10516 | NS2 |
| Human | HOXD8 | NP_062458 | 3234 | NS2 |
| Human | NR4A1 | NP_002126 | 3164 | NS2 |
| Human | POU3F2 | NP_005595 | 5454 | NS2 |
| Human | RPL11 | ENSP00000363676 | | NS2 |
| Human | RPN1 | ENSP00000296255 | | NS2 |
| Human | SETD2 | NP_054878 | 29072 | NS2 |
| Human | SMURF2 | ENSP00000262435 | | NS2 |
| Human | TRIM27 | NP_006501 | 5987 | NS2 |
| Human | sept-10 | NP_653311 | 151011 | NS3 |
| Human | A1BG | ENSP00000263100 | | NS3 |
| Human | ABCC3 | ENSP00000285238 | | NS3 |
| Human | ACTN1 | NP_001093 | 87 | NS3 |
| Human | ACTN2 | NP_001094 | 88 | NS3 |
| Human | AEBP1 | NP_001120 | 165 | NS3 |
| Human | AHCY | ENSP00000217426 | | NS3 |
| Human | AHSG | ENSP00000273784 | | NS3 |
| Human | ALB | ENSP00000370290 | | NS3 |
| Human | ANKRD12 | NP_056023 | 23253 | NS3 |
| Human | ANKRD28 | NP_056014 | 23243 | NS3 |
| Human | APOA1 | ENSP00000236850 | | NS3 |
| Human | APOA2 | ENSP00000356969 | | NS3 |
| Human | ARFIP2 | NP_036534 | 23647 | NS3 |
| Human | ARG1 | ENSP00000349446 | | NS3 |
| Human | ARHGDIA | ENSP00000269321 | | NS3 |
| Human | ARHGEF6 | NP_004831 | 9459 | NS3 |
| Human | ARNT | NP_001659 | 405 | NS3 |
| Human | ARS2 | NP_877952 | 51593 | NS3 |
| Human | ASXL1 | NP_056153 | 171023 | NS3 |
| Human | ATP5H | ENSP00000301587 | | NS3 |
| Human | AZGP1 | ENSP00000292401 | | NS3 |
| Human | B2M | NP_004039 | 567 | NS3 |
| Human | BCAN | NP_068767 | 63827 | NS3 |
| Human | BCKDK | NP_005872 | 10295 | NS3 |
| Human | BCL2A1 | NP_004040 | 597 | NS3 |
| Human | BCL6 | NP_001697 | 604 | NS3 |
| Human | BCR | ENSP00000335450 | | NS3 |
| Human | BZRAP1 | NP_004749 | 9256 | NS3 |
| Human | C10orf18 | XP_374765 | 54906 | NS3 |
| Human | C10orf18 | XP_943063 | 54906 | NS3 |
| Human | C10orf6 | NP_060591 | 55719 | NS3 |
| Human | C12orf41 | NP_060292 | 54934 | NS3 |
| Human | C14orf173 | NP_001026884 | 64423 | NS3 |
| Human | C16orf7 | NP_004904 | 9605 | NS3 |
| Human | C1orf165 | NP_078879 | 79656 | NS3 |
| Human | C1orf94 | NP_116273 | 84970 | NS3 |
| Human | C1S | ENSP00000328173 | | NS3 |
| Human | C9orf30 | NP_542386 | 91283 | NS3 |
| Human | CALCOCO2 | NP_005822 | 10241 | NS3 |
| Human | CAT | ENSP00000241052 | | NS3 |
| Human | CBY1 | NP_056188 | 25776 | NS3 |
| Human | CCDC21 | NP_001012524 | 64793 | NS3 |
| Human | CCDC37 | NP_872434 | 348807 | NS3 |
| Human | CCDC52 | NP_653319 | 152185 | NS3 |
| Human | CCDC66 | NP_001012524 | 285331 | NS3 |
| Human | CCDC95 | NP_775889 | 283899 | NS3 |
| Human | CCHCR1 | NP_061925 | 54535 | NS3 |
| Human | CCNDBP1 | ENSP00000349047 | | NS3 |
| Human | CD5L | NP_005885 | 922 | NS3 |
| Human | CDC23 | NP_004652 | 8697 | NS3 |
| Human | CELSR2 | NP_001399 | 1952 | NS3 |
| Human | CENPC1 | NP_001803 | 1060 | NS3 |
| Human | CEP152 | NP_055800 | 22995 | NS3 |
| Human | CEP192 | NP_115518 | 55125 | NS3 |
| Human | CES1 | ENSP00000353720 | | NS3 |
| Human | CFP | NP_002612 | 5199 | NS3 |
| Human | CHPF | NP_078812 | 79586 | NS3 |
| Human | COL3A1 | ENSP00000304408 | | NS3 |
| Human | CORO1B | NP_065174 | 57175 | NS3 |
| Human | COX3 | NP_536849 | 4514 | NS3 |
| Human | CSNK2B | NP_001311 | 1460 | NS3 |
| Human | CTGF | NP_001892 | 1490 | NS3 |
| Human | CTSD | ENSP00000236671 | | NS3 |
| Human | CTSF | ENSP00000310832 | | NS3 |
| Human | CXorf45 | NP_001034299 | 79868 | NS3 |
| Human | DEAF1 | NP_066288 | 10522 | NS3 |
| Human | DEDD2 | ENSP00000336972 | | NS3 |
| Human | DES | NP_001918 | 1674 | NS3 |
| Human | DLAT | NP_001922 | 1737 | NS3 |
| Human | DOCK7 | NP_212132 | 85440 | NS3 |
| Human | DPF1 | NP_004638 | 8193 | NS3 |
| Human | DPP7 | XP_001130451 | 29952 | NS3 |
| Human | ECHS1 | ENSP00000357535 | | NS3 |
| Human | EEF1A1 | NP_001393 | 1915 | NS3 |
| Human | EFEMP1 | NP_004096 | 2202 | NS3 |
| Human | EFEMP2 | NP_058634 | 30008 | NS3 |
| Human | EIF1 | NP_005792 | 10209 | NS3 |
| Human | EIF4ENIF1 | NP_062817 | 56478 | NS3 |
| Human | FAM120B | NP_115824 | 84498 | NS3 |
| Human | FAM62B | ENSP00000251527 | | NS3 |
| Human | FAM65A | NP_078795 | 79567 | NS3 |
| Human | FAM96B | ENSP00000299761 | | NS3 |
| Human | FBF1 | XP_951284 | 85302 | NS3 |
| Human | FBLN1 | NP_006477 | 2192 | NS3 |
| Human | FBLN1 | NP_001987 | 2192 | NS3 |
| Human | FBLN2 | NP_001989 | 2199 | NS3 |
| Human | FBLN2 | NP_001004019 | 2199 | NS3 |
| Human | FBLN5 | NP_006320 | 10516 | NS3 |
| Human | FBN1 | NP_000129 | 2200 | NS3 |
| Human | FBN3 | NP_115823 | 84467 | NS3 |
| Human | FES | NP_001996 | 2242 | NS3 |
| Human | FGA | ENSP00000306361 | | NS3 |
| Human | FGA | ENSP00000351465 | | NS3 |
| Human | FGB | ENSP00000306099 | | NS3 |
| Human | FIGNL1 | NP_071399 | 63979 | NS3 |
| Human | FLAD1 | NP_079483 | 80308 | NS3 |
| Human | FLJ11286 | NP_060851 | 55337 | NS3 |
| Human | FN1 | NP_002017 | 2335 | NS3 |
| Human | FRMPD4 | NP_055543 | 9758 | NS3 |
| Human | FRS3 | NP_006644 | 10817 | NS3 |
| Human | FTH1 | NP_002023 | 2495 | NS3 |
| Human | FUCA2 | NP_114409 | 2519 | NS3 |
| Human | GAA | NP_001073271 | 2548 | NS3 |
| Human | GBP2 | NP_004111 | 2634 | NS3 |
| Human | GC | ENSP00000273951 | | NS3 |
| Human | GFAP | NP_002046 | 2670 | NS3 |
| Human | GNB2 | NP_005264 | 2783 | NS3 |
| Human | GON4L | NP_001032622 | 54856 | NS3 |
| Human | HIVEP2 | NP_006725 | 3097 | NS3 |
| Human | HOMER3 | NP_004829 | 9454 | NS3 |
| Human | HP | ENSP00000348170 | | NS3 |
| Human | HTRA1 | ENSP00000357980 | | NS3 |
| Human | IFI44 | ENSP00000359783 | | NS3 |
| Human | IQWD1 | NP_060912 | 55827 | NS3 |
| Human | ITCH | ENSP00000363996 | | NS3 |
| Human | ITGB4 | NP_001005731 | 3691 | NS3 |
| Human | JAG2 | NP_002217 | 3714 | NS3 |
| Human | JUN | NP_002219 | 3725 | NS3 |
| Human | KHDRBS1 | NP_006550 | 10657 | NS3 |
| Human | KIAA1012 | ENSP00000348268 | | NS3 |
| Human | KIAA1549 | XP_371956 | 57670 | NS3 |
| Human | KIF17 | NP_065867 | 57576 | NS3 |
| Human | KIF7 | NP_940927 | 374654 | NS3 |
| Human | KNG1 | ENSP00000287611 | | NS3 |
| Human | KNG1 | ENSP00000265023 | | NS3 |
| Human | KPNA1 | NP_002255 | 3836 | NS3 |
| Human | KPNB1 | ENSP00000290158 | | NS3 |
| Human | L3MBTL3 | NP_115814 | 84456 | NS3 |
| Human | LAMA5 | NP_005551 | 3911 | NS3 |
| Human | LAMB2 | NP_002283 | 3913 | NS3 |
| Human | LAMC3 | NP_006050 | 10319 | NS3 |
| Human | LDB1 | NP_003884 | 8861 | NS3 |
| Human | LOC728302 | XP_001126546 | 728302 | NS3 |
| Human | LRRC7 | NP_065845 | 57554 | NS3 |
| Human | LRRCC1 | NP_001070969 | 85444 | NS3 |
| Human | LTBP4 | NP_003564 | 8425 | NS3 |
| Human | LZTS2 | NP_115805 | 84445 | NS3 |
| Human | MAGED1 | NP_008917 | 9500 | NS3 |
| Human | MAPK7 | NP_002740 | 5598 | NS3 |
| Human | MARCO | ENSP00000318916 | | NS3 |
| Human | MASP2 | ENSP00000366166 | | NS3 |
| Human | MEGF8 | NP_001401 | 1954 | NS3 |
| Human | MLLT4 | NP_005927 | 4301 | NS3 |
| Human | MLXIP | NP_055753 | 22877 | NS3 |
| Human | MORC4 | NP_078933 | 79710 | NS3 |
| Human | MORF4L1 | NP_996670 | 10933 | NS3 |
| Human | MPDZ | ENSP00000318809 | | NS3 |
| Human | MVP | NP_059447 | 9961 | NS3 |
| Human | MYL6 | ENSP00000293422 | | NS3 |
| Human | NAP1L1 | NP_004528 | 4673 | NS3 |
| Human | NCAN | NP_004377 | 1463 | NS3 |
| Human | NDC80 | NP_068798 | 10403 | NS3 |
| Human | NEFL | NP_006149 | 4747 | NS3 |
| Human | NEFM | NP_005373 | 4741 | NS3 |
| Human | NELL1 | NP_006148 | 4745 | NS3 |
| Human | NELL2 | NP_006150 | 4753 | NS3 |
| Human | NID1 | NP_002499 | 4811 | NS3 |
| Human | NID2 | NP_031387 | 22795 | NS3 |
| Human | NOTCH1 | NP_060087 | 4851 | NS3 |
| Human | N-PAC | NP_115958 | 84656 | NS3 |
| Human | NUCB1 | ENSP00000263273 | | NS3 |
| Human | NUP133 | ENSP00000355640 | | NS3 |
| Human | NUP62 | NP_036478 | 23636 | NS3 |
| Human | OBSCN | NP_443075 | 84033 | NS3 |
| Human | ORM1 | ENSP00000259396 | | NS3 |
| Human | OTC | ENSP00000039007 | | NS3 |
| Human | PARP2 | ENSP00000250416 | | NS3 |
| Human | PARP4 | NP_006428 | 143 | NS3 |
| Human | PCYT2 | NP_002852 | 5833 | NS3 |
| Human | PDE4DIP | NP_071754 | 9659 | NS3 |
| Human | PDLIM5 | NP_006448 | 10611 | NS3 |
| Human | PGM1 | ENSP00000342316 | | NS3 |
| Human | PICK1 | NP_036539 | 9463 | NS3 |
| Human | PKNOX1 | NP_004562 | 5316 | NS3 |
| Human | PLEKHG4 | NP_056247 | 25894 | NS3 |
| Human | PNPLA8 | NP_056538 | 50640 | NS3 |
| Human | PNPT1 | ENSP00000260604 | | NS3 |
| Human | POLDIP2 | ENSP00000003607 | | NS3 |
| Human | PRG4 | ENSP00000356452 | | NS3 |
| Human | PRRC1 | NP_570721 | 133619 | NS3 |
| Human | PSMA6 | ENSP00000261479 | | NS3 |
| Human | PSMB9 | NP_002791 | 5698 | NS3 |
| Human | PSME3 | NP_005780 | 10197 | NS3 |
| Human | PTPRF | ENSP00000361479 | | NS3 |
| Human | PTPRN2 | NP_002838 | 5799 | NS3 |
| Human | RABEP1 | NP_004694 | 9135 | NS3 |
| Human | RAI14 | NP_056392 | 26064 | NS3 |
| Human | RASAL2 | NP_004832 | 9462 | NS3 |
| Human | RBM4 | NP_002887 | 5936 | NS3 |
| Human | RCN3 | NP_065701 | 57333 | NS3 |
| Human | RGNEF | XP_942978 | 64283 | NS3 |
| Human | RICS | NP_055530 | 9743 | NS3 |
| Human | RING1 | ENSP00000363787 | | NS3 |
| Human | RINT1 | NP_068749 | 60561 | NS3 |
| Human | RLF | ENSP00000361857 | | NS3 |
| Human | RNF31 | NP_060469 | 55072 | NS3 |
| Human | ROGDI | NP_078865 | 79641 | NS3 |
| Human | RP11-130N24.1 | NP_001008537 | 340533 | NS3 |
| Human | RSHL2 | NP_114130 | 83861 | NS3 |
| Human | RUSC2 | NP_055621 | 9853 | NS3 |
| Human | SBF1 | NP_002963 | 6305 | NS3 |
| Human | SDCCAG8 | NP_006633 | 10806 | NS3 |
| Human | SECISBP2 | NP_076982 | 79048 | NS3 |
| Human | SELO | ENSP00000248845 | | NS3 |
| Human | SERTAD1 | NP_037508 | 29950 | NS3 |
| Human | SESTD1 | NP_835224 | 91404 | NS3 |
| Human | SF3B2 | NP_006833 | 10992 | NS3 |
| Human | SGCB | ENSP00000295211 | | NS3 |
| Human | SIAH1 | NP_001006611 | 6477 | NS3 |
| Human | SLIT1 | NP_003052 | 6585 | NS3 |
| Human | SLIT2 | NP_004778 | 9353 | NS3 |
| Human | SLIT3 | NP_003053 | 6586 | NS3 |
| Human | SMARCE1 | ENSP00000264640 | | NS3 |
| Human | SMURF2 | NP_073576 | 64750 | NS3 |
| Human | SNX4 | NP_003785 | 8723 | NS3 |
| Human | SPOCK3 | NP_058646 | 50859 | NS3 |
| Human | SPON1 | NP_006099 | 10418 | NS3 |
| Human | SPP2 | ENSP00000168148 | | NS3 |
| Human | SRPX2 | NP_055282 | 27286 | NS3 |
| Human | SSX2IP | NP_054740 | 117178 | NS3 |
| Human | STAB1 | NP_055951 | 23166 | NS3 |
| Human | STAT3 | NP_003141 | 6774 | NS3 |
| Human | STRAD | ENSP00000245865 | | NS3 |
| Human | SVEP1 | NP_699197 | 79987 | NS3 |
| Human | SYNE1 | NP_056108 | 23345 | NS3 |
| Human | SYNPO2 | XP_947873 | 171024 | NS3 |
| Human | SYNPO2 | XP_941429 | 171024 | NS3 |
| Human | TAF1 | NP_004597 | 6872 | NS3 |
| Human | TAF15 | ENSP00000309558 | | NS3 |
| Human | TBC1D2B | NP_055894 | 23102 | NS3 |
| Human | TBN | ENSP00000312792 | | NS3 |
| Human | TBXAS1 | NP_001052 | 6916 | NS3 |
| Human | TF | ENSP00000264998 | | NS3 |
| Human | TGFB1I1 | NP_001035919 | 7041 | NS3 |
| Human | TH1L | ENSP00000217129 | | NS3 |
| Human | THAP1 | NP_060575 | 55145 | NS3 |
| Human | TMEM63B | NP_060896 | 55362 | NS3 |
| Human | TPST2 | ENSP00000339813 | | NS3 |
| Human | TPT1 | ENSP00000339051 | | NS3 |
| Human | TRIM23 | NP_001647 | 373 | NS3 |
| Human | TRIM27 | NP_006501 | 5987 | NS3 |
| Human | TRIO | NP_009049 | 7204 | NS3 |
| Human | TRIP11 | NP_004230 | 9321 | NS3 |
| Human | TXNDC11 | NP_056998 | 51061 | NS3 |
| Human | UBE1C | NP_003959 | 9039 | NS3 |
| Human | USHBP1 | NP_114147 | 83878 | NS3 |
| Human | UXT | NP_705582 | 8409 | NS3 |
| Human | VCAN | NP_004376 | 1462 | NS3 |
| Human | VIM | NP_003371 | 7431 | NS3 |
| Human | VWF | NP_000543 | 7450 | NS3 |
| Human | WDTC1 | ENSP00000355317 | | NS3 |
| Human | XAB2 | NP_064581 | 56949 | NS3 |
| Human | XRN2 | NP_036387 | 22803 | NS3 |
| Human | YY1AP1 | NP_620829 | 55249 | NS3 |
| Human | ZADH1 | ENSP00000267568 | | NS3 |
| Human | ZBTB1 | NP_055765 | 22890 | NS3 |
| Human | ZCCHC7 | NP_115602 | 84186 | NS3 |
| Human | ZHX3 | NP_055850 | 23051 | NS3 |
| Human | ZMYM2 | NP_003444 | 7750 | NS3 |
| Human | ZNF281 | NP_036614 | 23528 | NS3 |
| Human | ZNF410 | NP_067011 | 57862 | NS3 |
| Human | ZNF440 | ENSP00000305373 | | NS3 |
| Human | ZXDC | ENSP00000374359 | | NS3 |
| Human | ZZZ3 | NP_056349 | 26009 | NS3 |
| Human | APOA1 | ENSP00000236850 | | NS3/4A |
| Human | DNAJB1 | ENSP00000254322 | | NS3/4A |
| Human | CREB3 | NP_006359 | 10488 | NS4A |
| Human | ELAC2 | NP_060597 | 60528 | NS4A |
| Human | HOXD8 | NP_062458 | 3234 | NS4A |
| Human | NR4A1 | NP_002126 | 3164 | NS4A |
| Human | TRAF3IP3 | NP_079504 | 80342 | NS4A |
| Human | UBQLN1 | NP_038466 | 29979 | NS4A |
| Human | APOA1 | ENSP00000236850 | | NS4B |
| Human | ATF6 | ENSP00000356919 | | NS4B |
| Human | KNG1 | ENSP00000287611 | | NS4B |
| Human | NR4A1 | NP_002126 | 3164 | NS4B |
| Human | AARS2 | ENSP00000244571 | | NS5A |
| Human | ABCC3 | ENSP00000285238 | | NS5A |
| Human | ACLY | NP_001087 | 47 | NS5A |
| Human | ACTB | ENSP00000349960 | | NS5A |
| Human | ALDOB | ENSP00000363988 | | NS5A |
| Human | APOB | ENSP00000370431 | | NS5A |
| Human | ARFIP1 | NP_055262 | 27236 | NS5A |
| Human | ARFIP1 | NP_001020766 | 27236 | NS5A |
| Human | ASXL1 | ENSP00000364839 | | NS5A |
| Human | AXIN1 | NP_003493 | 8312 | NS5A |
| Human | C10orf30 | NP_689964 | 222389 | NS5A |
| Human | C9orf23 | ENSP00000368242 | | NS5A |
| Human | CADPS | NP_003707 | 8618 | NS5A |
| Human | CADPS2 | NP_001009571 | 93664 | NS5A |
| Human | CCDC100 | NP_694955 | 153241 | NS5A |
| Human | CCDC90A | ENSP00000368468 | | NS5A |
| Human | CCT7 | ENSP00000258091 | | NS5A |
| Human | CEP250 | NP_009117 | 11190 | NS5A |
| Human | CEP63 | NP_079456 | 80254 | NS5A |
| Human | CES1 | ENSP00000353720 | | NS5A |
| Human | CFH | ENSP00000352658 | | NS5A |
| Human | COL3A1 | ENSP00000304408 | | NS5A |
| Human | DDX5 | ENSP00000225792 | | NS5A |
| Human | DNAJA3 | NP_005138 | 9093 | NS5A |
| Human | EFEMP1 | NP_004096 | 2202 | NS5A |
| Human | EIF3S2 | ENSP00000362688 | | NS5A |
| Human | ETFA | ENSP00000267950 | | NS5A |
| Human | FGB | ENSP00000306099 | | NS5A |
| Human | FHL2 | NP_963849 | 2274 | NS5A |
| Human | GLTSCR2 | ENSP00000246802 | | NS5A |
| Human | GOLGA2 | NP_004477 | 2801 | NS5A |
| Human | GPS2 | NP_004480 | 2874 | NS5A |
| Human | HRSP12 | ENSP00000254878 | | NS5A |
| Human | IGLL1 | NP_064455 | 3537 | NS5A |
| Human | ITGAL | NP_002200 | 3683 | NS5A |
| Human | LDHD | ENSP00000300051 | | NS5A |
| Human | LIMS2 | NP_060450 | 55679 | NS5A |
| Human | LOC374395 | NP_955369 | 374395 | NS5A |
| Human | MAF | ENSP00000327048 | | NS5A |
| Human | MBD4 | ENSP00000249910 | | NS5A |
| Human | MKRN2 | ENSP00000373551 | | NS5A |
| Human | MOBK1B | NP_060691 | 55233 | NS5A |
| Human | MON2 | ENSP00000261188 | | NS5A |
| Human | NAP1L1 | NP_004528 | 4673 | NS5A |
| Human | NFE2 | NP_006154 | 4778 | NS5A |
| Human | NUCB1 | NP_006175 | 4924 | NS5A |
| Human | OS9 | ENSP00000373799 | | NS5A |
| Human | OS9 | ENSP00000373798 | | NS5A |
| Human | PARVG | NP_071424 | 64098 | NS5A |
| Human | PMVK | NP_006547 | 10654 | NS5A |
| Human | POMP | ENSP00000370205 | | NS5A |
| Human | PPP1R13L | NP_006654 | 10848 | NS5A |
| Human | PSMB8 | ENSP00000364016 | | NS5A |
| Human | PSMB9 | NP_002791 | 5698 | NS5A |
| Human | RLF | ENSP00000361857 | | NS5A |
| Human | RPL18A | NP_000971 | 6142 | NS5A |
| Human | RRBP1 | NP_001036041 | 6238 | NS5A |
| Human | SHARPIN | NP_112236 | 81858 | NS5A |
| Human | SMYD3 | NP_073580 | 64754 | NS5A |
| Human | SORBS2 | NP_066547 | 8470 | NS5A |
| Human | SORBS3 | NP_005766 | 10174 | NS5A |
| Human | THBS1 | NP_003237 | 7057 | NS5A |
| Human | TMF1 | NP_009045 | 7110 | NS5A |
| Human | TP53BP2 | NP_005417 | 7159 | NS5A |
| Human | TRIOBP | NP_001034230 | 11078 | NS5A |
| Human | TST | ENSP00000249042 | | NS5A |
| Human | TXNDC11 | NP_056998 | 51061 | NS5A |
| Human | UBASH3A | NP_061834 | 53347 | NS5A |
| Human | UBC | ENSP00000344818 | | NS5A |
| Human | USP19 | NP_006668 | 10869 | NS5A |
| Human | VPS52 | NP_072047 | 6293 | NS5A |
| Human | ZGPAT | ENSP00000332013 | | NS5A |
| Human | ZH2C2 | NP_060146 | 54826 | NS5A |
| Human | ZNF135 | ENSP00000346852 | | NS5A |
| Human | ZNF350 | ENSP00000243644 | | NS5A |
| Human | ZNF646 | NP_055514 | 9726 | NS5A |
| Human | ZNHIT1 | ENSP00000304593 | | NS5A |
| Human | ZNHIT4 | ENSP00000233331 | | NS5A |
| Human | APOA1 | ENSP00000236850 | | NS5B |
| Human | APOC3 | ENSP00000364494 | | NS5B |
| Human | CCNDBP1 | ENSP00000349047 | | NS5B |
| Human | CEP250 | NP_009117 | 11190 | NS5B |
| Human | CEP68 | NP_055962 | 23177 | NS5B |
| Human | CTSF | ENSP00000310832 | | NS5B |
| Human | HOXD8 | NP_062458 | 3234 | NS5B |
| Human | MGC2752 | NP_076428 | 65996 | NS5B |
| Human | MOBK1B | NP_060691 | 55233 | NS5B |
| Human | OS9 | NP_001017956 | 10956 | NS5B |
| Human | OTC | ENSP00000039007 | | NS5B |
| Human | PKM2 | NP_002645 | 5315 | NS5B |
| Human | PSMB9 | NP_002791 | 5698 | NS5B |
| Human | SETD2 | NP_054878 | 29072 | NS5B |
| Human | SHARPIN | NP_112236 | 81858 | NS5B |
| Human | TAGLN | ENSP00000278968 | | NS5B |
| Human | TUBB2C | NP_006079 | 10383 | NS5B |
| Human | CREB3 | NP_006359 | 10488 | p7 |
| Human | FBLN2 | NP_001989 | 2199 | p7 |
| Human | FXYD6 | NP_071286 | 53826 | p7 |
| Human | LMNB1 | NP_005564 | 4001 | p7 |
| Human | RNUXA | ENSP00000297540 | | p7 |
| Human | SLC39A8 | ENSP00000349174 | | p7 |
| Human | SLIT2 | NP_004778 | 9353 | p7 |
| Human | UBQLN1 | NP_038466 | 29979 | p7 |
| Human | UBQLN4 | NP_064516 | 56893 | p7 |

### Screening Methods

The invention relates to methods for screening compounds for treating and/or preventing an HCV infection comprising the steps of:

a) determining the ability of a candidate compound to inhibit the interaction between a viral HCV protein and a human protein as described above, and
b) selecting the candidate compound that inhibits said interaction between said viral protein and said human protein.

In one embodiment the step b) consists in generating physical values which illustrate or not the ability of said candidate compound to inhibit the interaction between said HCV protein and said human protein and comparing said values with standard physical values obtained in the same assay performed in the absence of the said candidate compound. The “physical values” that are referred to above may be of various kinds depending of the binding assay that is performed, but notably encompass light absorbance values, radioactive signals and intensity value of fluorescence signal. If after the comparison of the physical values with the standard physical values, it is determined that the said candidate compound inhibits the binding between said HCV protein and said human protein, then the candidate is positively selected at step b).

The compounds that inhibit the interaction between the HCV protein and human protein encompass those compounds that bind either to HCV protein or to human protein, provided that the binding of the said compounds of interest then prevents the interaction between HCV protein and human protein.

### Labelled Polypeptides

In one embodiment, any protein of the invention is labelled with a detectable molecule.

According to the invention, said detectable molecule may consist of any compound or substance that is detectable by spectroscopic, photochemical, biochemical, immunochemical or chemical means. For example, useful detectable molecules include radioactive substance (including those comprising ³²P, ²⁵S, ³H, or ¹²⁵I), fluorescent dyes (including 5-bromodesosyrudin, fluorescein, acetylaminofluorene or digoxigenin), fluorescent proteins (including GFPs and YFPs), or detectable proteins or peptides (including biotin, polyhistidine tails or other antigen tags like the HA antigen, the FLAG antigen, the c-myc antigen and the DNP antigen).

According to the invention, the detectable molecule is located at, or bound to, an amino acid residue located outside the said amino acid sequence of interest, in order to minimise or prevent any artefact for the binding between said polypeptides or between the candidate compound and or any of said polypeptides.

In another particular embodiment, the polypeptides of the invention are fused with a GST tag (Glutathione S-transferase). In this embodiment, the GST moiety of the said fusion protein may be used as detectable molecule. In the said fusion protein, the GST may be located either at the N-terminal end or at the C-terminal end. The GST detectable molecule may be detected when it is subsequently brought into contact with an anti-GST antibody, including with a labelled anti-GST antibody. Anti-GST antibodies labelled with various detectable molecules are easily commercially available.

In another particular embodiment, proteins of the invention are fused with a poly-histidine tag. Said poly-histidine tag usually comprises at least four consecutive hisitidine residues and generally at least six consecutive histidine residues. Such a polypeptide tag may also comprise up to 20 consecutive histidine residues. Said poly-histidine tag may be located either at the N-terminal end or at the C-terminal end In this embodiment, the poly-histidine tag may be detected when it is subsequently brought into contact with an anti-poly-histidine antibody, including with a labelled anti-poly-histidine antibody. Anti-poly-histidine antibodies labelled with various detectable molecules are easily commercially available.

In a further embodiment, the proteins of the invention are fused with a protein moiety consisting of either the DNA binding domain or the activator domain of a transcription factor. Said protein moiety domain of transcription may be located either at the N-terminal end or at the C-terminal end. Such a DNA binding domain may consist of the well-known DNA binding domain of LexA protein originating form E. Coli. Moreover said activator domain of a transcription factor may consist of the activator domain of the well-known Gal4 protein originating from yeast.

### Two-Hybrid Assay

In one embodiment of the screening method according to the invention, the proteins of the invention comprise a portion of a transcription factor. In said assay, the binding together of the first and second portions generates a functional transcription factor that binds to a specific regulatory DNA sequence, which in turn induces expression of a reporter DNA sequence, said expression being further detected and/or measured. A positive detection of the expression of said reporter DNA sequence means that an active transcription factor is formed, due to the binding together of said first HCV protein and second human protein.

Usually, in a two-hybrid assay, the first and second portion of a transcription factor consist respectively of (i) the DNA binding domain of a transcription factor and (ii) the activator domain of a transcription factor. In some embodiments, the DNA binding domain and the activator domain both originate from the same naturally occurring transcription factor. In some embodiments, the DNA binding domain and the activator domain originate from distinct naturally occurring factors, while, when bound together, these two portions form an active transcription factor. The term “portion” when used herein for transcription factor, encompass complete proteins involved in multi protein transcription factors, as well as specific functional protein domains of a complete transcription factor protein.

Therefore in one embodiment of the invention, step a) of the screening method of the invention comprises the following steps:
(1) providing a host cell expressing:
   a first fusion polypeptide between (i) a HCV protein as defined above and (ii) a first protein portion of transcription factor
   a second fusion polypeptide between (i) a human protein as defined above and (ii) a second portion of a transcription factor
said transcription factor being active on DNA target regulatory sequence when the first and second protein portion are bound together and
said host cell also containing a nucleic acid comprising (i) a regulatory DNA sequence that may be activated by said active transcription factor and (ii) a DNA report sequence that is operatively linked to said regulatory sequence
(2) bringing said host cell provided at step 1) into contact with a candidate compound to be tested
(3) determining the expression level of said DNA reporter sequence

The expression level of said DNA reporter sequence that is determined at step (3) above is compared with the expression of said DNA reporter sequence when step (2) is omitted. A lower expression level of said DNA reporter sequence in the presence of the candidate compound means that the said candidate compound effectively inhibits the binding between HCV protein and human protein and that said candidate compound may be positively selected a step b) of the screening method.

Suitable host cells include, without limitation, prokaryotic cells (such as bacteria) and eukaryotic cells (such as yeast cells, mammalian cells, insect cells, plant cells, etc.). However preferred host cell are yeast cells and more preferably a Saccharomyces cerevisiae cell or a Schizosaccharomyces pombe cell.

Similar systems of two-hybrid assays are well know in the art and therefore can be used to perform the screening method according to the invention (see. Fields et al. 1989; Vasavada et al. 1991; Fearon et al. 1992; Dang et al., 1991, Chien et al. 1991, U.S. Pat. No. 5,283,173, U.S. Pat. No. 5,667,973, U.S. Pat. No. 5,468,614, U.S. Pat. No. 5,525,490 and U.S. Pat. No. 5,637,463). For instance, as described in these documents, the Gal4 activator domain can be used for performing the screening method according to the invention. Gal4 consists of two physically discrete modular domains, one acting as the DNA binding domain, the other one functioning as the transcription-activation domain. The yeast expression system described in the foregoing documents takes advantage of this property. The expression of a Gal1-LacZ reporter gene under the control of a Gal4-activated promoter depends on the reconstitution of Gal4 activity via protein-protein interaction. Colonies containing interacting polypeptides are detected with a chromogenic substrate for β-galactosidase. A compete kit (MATCHMAKER™) for identifying protein-protein interactions is commercially available from Clontech. So in one embodiment, a first HCV protein as above defined is fused to the DNA binding domain of Gal4 and the second human protein as above defined is fused to the activation domain of Gal4.

The expression of said detectable marker gene may be assessed by quantifying the amount of the corresponding specific mRNA produced. However, usually the detectable marker gene sequence encodes for detectable protein, so that the expression level of the said detectable marker gene is assessed by quantifying the amount of the corresponding protein produced. Techniques for quantifying the amount of mRNA or protein are well known in the art. For example, the detectable marker gene placed under the control of regulatory sequence may consist of the β-galactosidase as above described.

### Western Blotting

In another one embodiment, step a) comprises a step of subjecting to a gel migration assay the mixture of the first HCV protein and the second human protein as above defined, with or without the candidate compound to be tested and then measuring the binding of the said polypeptides altogether by performing a detection of the complexes formed between said polypeptides. The gel migration assay can be carried out as known by the one skilled in the art.

Therefore in one embodiment of the invention, step a) of the screening method of the invention comprises the following steps:
(1) providing a first HCV protein and a second human protein as defined above
(2) bringing into contact the candidate compound to be tested with said polypeptides
(3) performing a gel migration assay a suitable migration substrate with said polypeptides and said candidate compound as obtained at step (2)
(4) detecting and quantifying the complexes formed between said polypeptides on the migration assay as performed at step (3).

The presence or the amount of the complexes formed between the proteins are then compared with the results obtained when the assay is performed in the absence of the candidate compound to be tested. Therefore, when no complexes between the proteins is detected or, alternatively when those complexes are present in a lower amount compared to the amount obtained in the absence of the candidate compound, then the candidate compound may be positively selected at step b) of the screening method.

The detection of the complexes formed between the said two proteins may be easily performed by staining the migration gel with a suitable dye and then determining the protein bands corresponding to the protein analysed since the complexes formed between the first and the second proteins possess a specific apparent molecular weight. Staining of proteins in gels may be done using the standard Coomassie brilliant blue (or PAGE blue), Amido Black, or silver stain reagents of different kinds. Suitable gels are well known in the art such as sodium dodecyl (lauryl) sulfate-polyacrylamide gel. In a general manner, western blotting assays are well known in the art and have been widely described (Rybicki et al., 1982; Towbin et al. 1979; Kurien et al. 2006).

In a particular embodiment, the protein bands corresponding to the proteins submitted to the gel migration assay can be detected by specific antibodies. It may used both antibodies directed against the HCV proteins and antibodies specifically directed against the human proteins.

In another embodiment, the said two proteins are labelled with a detectable antigen as above described. Therefore, the proteins bands can be detected by specific antibodies directed against said detectable antigen. Preferably, the detectable antigen conjugates to the HCV protein is different from the antigen conjugated to the human protein. For instance, the first HCV protein can be fused to a GST detectable antigen and the second human protein can be fused with the HA antigen. Then the protein complexes formed between the two proteins may be quantified and determined with antibodies directed against the GST and HA antigens respectively.

### Biosensor Assays

In another embodiment, step a) included the use of an optical biosensor such as described by Edwards et al. (1997) or also by Szabo et al. (1995). This technique allows the detection of interactions between molecules in real time, without the need of labelled molecules. This technique is indeed bases on the surface plasmon resonance (SPR) phenomenon. Briefly, a first protein partner is attached to a surface (such as a carboxymethyl dextran matrix). Then the second protein partner is incubated with the previously immobilised first partner, in the presence or absence of the candidate compound to be tested. Then the binding including the binding level or the absence of binding between said protein partners is detected. For this purpose, a light beam is directed towards the side of the surface area of the substrate that does not contain the sample to be tested and is reflected by said surface. The SPR phenomenon causes a decrease in the intensity of the reflected light with a combination of angle and wavelength. The binding of the first and second protein partner causes a change in the refraction index on the substrate surface, which change is detected as a change in the SPR signal.

### Affinity Chromatography

In another one embodiment of the invention, the screening method includes the use of affinity chromatography.

Candidate compounds for use in the screening method above can also be selected by any immunoaffinity chromatography technique using any chromatographic substrate onto which (i) the first HCV protein or (ii) the second human protein as above defined, has previously been immobilised, according to techniques well known from the one skilled in the art. Briefly, the HCV protein or the human protein as above defined, may be attached to a column using conventional techniques including chemical coupling to a suitable column matrix such as agarose, Affi Gel®, or other matrices familiar to those of skill in the art. In some embodiment of this method, the affinity column contains chimeric proteins in which the HCV protein or human protein as above defined, is fused to glutathion—s-transferase (GST). Then a candidate compound is brought into contact with the chromatographic substrate of the affinity column previously, simultaneously or subsequently to the other protein among the said first and second protein. The after washing, the chromatography substrate is eluted and the collected elution liquid is analysed by detection and/or quantification of extent, the candidate compound has impaired the binding between (i) first HCV protein and (ii) the second human protein.

### Fluorescence Assays

In another one embodiment of the screening method according to the invention, the first HCV protein and the second human protein as above defined are labelled with a fluorescent molecule or substrate. Therefore, the potential alteration effect of the candidate compound to be tested on the binding between the first HCV protein and the second human protein as above defined is determined by fluorescence quantification.

For example, the first HCV protein and the second human protein as above defined may be fused with auto-fluorescent polypeptides, as GFP or YFPs as above described. The first HCV protein and the second human protein as above defined may also be labelled with fluorescent molecules that are suitable for performing fluorescence detection and/or quantification for the binding between said proteins using fluorescence energy transfer (FRET) assay. The first HCV protein and the second human protein as above defined may be directly labelled with fluorescent molecules, by covalent chemical linkage with the fluorescent molecule as GFP or YFP. The first HCV protein and the second human protein as above defined may also be indirectly labelled with fluorescent molecules, for example, by non covalent linkage between said polypeptides and said fluorescent molecule. Actually, said first HCV protein and second human protein as above defined may be fused with a receptor or ligand and said fluorescent molecule may be fused with the corresponding ligand or receptor, so that the fluorescent molecule can non-covalently bind to said first HCV protein and second human protein. A suitable receptor/ligand couple may be the biotin/streptavidin paired member or may be selected among an antigen/antibody paired member. For example, a protein according to the invention may be fused to a poly-histidine tail and the fluorescent molecule may be fused with an antibody directed against the poly-histidine tail.

As already specified, step a) of the screening method according to the invention encompasses determination of the ability of the candidate compound to inhibit the interaction between the HCV protein and the human protein as above defined by fluorescence assays using FRET. Thus, in a particular embodiment, the first HCV protein as above defined is labelled with a first fluorophore substance and the second human protein is labelled with a second fluorophore substance. The first fluorophore substance may have a wavelength value that is substantially equal to the excitation wavelength value of the second fluorophore, whereby the bind of said first and second proteins is detected by measuring the fluorescence signal intensity emitted at the emission wavelength of the second fluorophore substance. Alternatively, the second fluorophore substance may also have an emission wavelength value of the first fluorophore, whereby the binding of said and second proteins is detected by measuring the fluorescence signal intensity emitted at the wavelength of the first fluorophore substance.

The fluorophores used may be of various suitable kinds, such as the well-known lanthanide chelates. These chelates have been described as having chemical stability, long-lived fluorescence (greater than 0.1 ms lifetime) after bioconjugation and significant energy-transfer in specificity bioaffinity assay. Document U.S. Pat. No. 5,162,508 discloses bipyridine cryptates. Polycarboxylate chelators with TEKES type photosensitizers (EP0203047A1) and terpyridine type photosensitizers (EP0649020A1) are known. Document WO96/00901 discloses diethylenetriaminepentaacetic acid (DPTA) chelates which used carbostyril as sensitizer. Additional DPT chelates with other sensitizer and other tracer metal are known for diagnostic or imaging uses (e.g., EP0450742A1).

In a preferred embodiment, the fluorescence assay performed at step a) of the screening method consists of a Homogeneous Time Resolved Fluorescence (HTRF) assay, such as described in document WO 00/01663 or U.S. Pat. No. 6,740,756, the entire content of both documents being herein incorporated by reference. HTRF is a TR-FRET based technology that uses the principles of both TRF (time-resolved fluorescence) and FRET. More specifically, the one skilled in the art may use a HTRF assay based on the time-resolved amplified cryptate emission (TRACE) technology as described in Leblanc et al. (2002). The HTRF donor fluorophore is Europium Cryptate, which has the long-lived emissions of lanthanides coupled with the stability of cryptate encapsulation. XL665, a modified allophycocyanin purified from red algae, is the HTRF primary acceptor fluorophore. When these two fluorophores are brought together by a biomolecular interaction, a portion of the energy captured by the Cryptate during excitation is released through fluorescence emission at 620 nm, while the remaining energy is transferred to XL665. This energy is then released by XL665 as specific fluorescence at 665 nm. Light at 665 nm is emitted only through FRET with Europium. Because Europium Cryptate is always present in the assay, light at 620 nm is detected even when the biomolecular interaction does not bring XL665 within close proximity.

Therefore in one embodiment, step a) of the screening method may therefore comprises the steps of:
(1) bringing into contact a pre-assay sample comprising:
   a first HCV protein fused to a first antigen,
   a second human protein fused to a second antigen,
   a candidate compound to be tested;
(2) adding to the said pre assay sample of step (1):
   at least one antibody labelled with a European Cryptate which is specifically directed against the first said antigen,
   at least one antibody labelled with XL665 directed against the second said antigen;
(3) illuminating the assay sample of step (2) at the excitation wavelength of the said European Cryptate;
(4) detecting and/or quantifying the fluorescence signal emitted at the XL665 emission wavelength;
(5) comparing the fluorescence signal obtained at step (4) to the fluorescence obtained wherein pre assay sample of step (1) is prepared in the absence of the candidate compound to be tested.

If at step (5) as above described, the intensity value of the fluorescence signal is lower than the intensity value of the fluorescence signal found when pre assay sample of step (1) is prepared in the absence of the candidate compound to be tested, then the candidate compound may be positively selected at step b) of the screening method.

Antibodies labelled with a European Cryptate or labelled with XL665 can be directed against different antigens of interest including GST, poly-histidine tail, DNP, c-myx, HA antigen and FLAG which include. Such antibodies encompass those which are commercially available from CisBio (Bedfors, Mass., USA), and notably those referred to as 61 GSTKLA or 61 HISKLB respectively.

### Candidate Compounds

According to a one embodiment of the invention, the candidate compound of the invention may be selected from a library of compounds previously synthesised, or a library of compounds for which the structure is determined in a database, or from a library of compounds that have been synthesised de novo.

The candidate compound may be selected from the group of (a) proteins or peptides, (b) nucleic acids and (c) organic or chemical compounds. Illustratively, libraries of pre-selected candidate nucleic acids may be obtained by performing the SELEX method as described in documents U.S. Pat. No. 5,475,096 and U.S. Pat. No. 5,270,163. Further illustratively, the candidate compound may be selected from the group of antibodies directed against said HCV protein and said human proteins as above described.

### In Vivo Screening Methods

The candidate compounds that have been positively selected at the end of any one of the embodiments of the in vitro screening which has been described previously in the present specification may be subjected to further selection steps in view of further assaying its anti-HCV biological properties.

### Production of Proteins of the Invention

Proteins of the invention may be produced by any technique known per se in the art, such as without limitation, any chemical, biological, genetic or enzymatic technique, either alone or in combination(s).

Knowing the amino acid sequence of the desired sequence, one skilled in the art can readily produce said proteins, by standard techniques. For instance, they can be synthesized using well-known solid phase method, preferably using a commercially available peptide synthesis apparatus (such as that made by Applied Biosystems, Foster City, Calif.) and following the manufacturer's instructions.

Alternatively, the proteins of the invention can be synthesized by recombinant DNA techniques as is now well-known in the art. For example, these fragments can be obtained as DNA expression products after incorporation of DNA sequences encoding the desired proteins into expression vectors and introduction of such vectors into suitable eukaryotic or prokaryotic hosts that will express the desired proteins, from which they can be later isolated using well-known techniques.

A wide variety of host/expression vector combinations are employed in expressing the nucleic acids encoding for the polypeptides of the present invention. Useful expression vectors that can be used include, for example, segments of chromosomal, non-chromosomal and synthetic DNA sequences. Suitable vectors include, but are not limited to, derivatives of SV40 and pcDNA and known bacterial plasmids such as col EI, pCR1, pBR322, pMal-C2, pET, pGEX, pMB9 and derivatives thereof, plasmids such as RP4, phage DNAs such as the numerous derivatives of phage I such as NM989, as well as other phage DNA such as M13 and filamentous single stranded phage DNA; yeast plasmids such as the 2 microns plasmid or derivatives of the 2 microns plasmid, as well as centomeric and integrative yeast shuttle vectors; vectors useful in eukaryotic cells such as vectors useful in insect or mammalian cells; vectors derived from combinations of plasmids and phage DNAs, such as plasmids that have been modified to employ phage DNA or the expression control sequences; and the like.

Consequently, mammalian and typically human cells, as well as bacterial, yeast, fungi, insect, nematode and plant cells an used in the present invention and may be transfected by the nucleic acid or recombinant vector as defined herein. Examples of suitable cells include, but are not limited to, VERO cells, HELA cells such as ATCC No. CCL2, CHO cell lines such as ATCC No. CCL61, COS cells such as COS-7 cells and ATCC No. CRL 1650 cells, W138, BHK, HepG2, 3T3 such as ATCC No. CRL6361, A549, PC12, K562 cells, 293T cells, Sf9 cells such as ATCC No. CRL1711 and Cv1 cells such as ATCC No. CCL70. Other suitable cells that can be used in the present invention include, but are not limited to, prokaryotic host cells strains such as Escherichia coli, (e.g., strain DH5-[alpha]), Bacillus subtilis, Salmonella typhimurium, or strains of the genera of Pseudomonas, Streptomyces and Staphylococcus. Further suitable cells that can be used in the present invention include yeast cells such as those of Saccharomyces such as Saccharomyces cerevisiae.

### Therapeutic Methods and Uses

In a further aspect, the invention provides a method for treating an HCV infection or preventing an HCV infection comprising administering a subject in need thereof with a therapeutically effective amount of a compound that inhibits the interaction between the HCV and human proteins as described above. Said compound may be identified by the screening methods of the invention.

In the context of the invention, the term “treating” or “treatment”, as used herein, means reversing, alleviating, inhibiting the progress of, or preventing the disorder or condition to which such term applies, or one or more symptoms of such disorder or condition such as liver injury, metabolic disorders associated with hepatic steatosis, fibrogenesis and insulin resistance.

According to the invention, the term “patient” or “subject in need thereof”, is intended for a human or non-human mammal affected or likely to be affected with an HCV infection.

By a “therapeutically effective amount” of the compound of the invention is meant a sufficient amount of compound to treat an infection with HCV, at a reasonable benefit/risk ratio applicable to any medical treatment. It will be understood, however, that the total daily usage of the compound of the invention and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than those required to achieve the desired therapeutic effect and to gradually increase the dosage until the desired effect is achieved.

In another one embodiment the invention relates to the use of at least one compound that inhibits the interaction between the HCV and human proteins as described above for the manufacture of a medicament intended for treating an HCV infection or preventing an HCV infection.

The compound that inhibits the interaction between the HCV and human proteins as described above may be combined with pharmaceutically acceptable excipients. “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The invention will further be illustrated in view of the following figures and examples.

### FIGURES

FIG. 1: The HCV Interaction Network.

A. Nomenclature.

V: viral protein (black node). H_{HCV}: human protein interacting with HCV proteins (red node). H_{NOT-HCV}: human protein not interacting with HCV proteins (blue node). V-H_{HCV}: HCV-human protein interaction (red edge). H_{HCV}-H_{HCV}: interaction between HCV-interacting human proteins (blue edge). H-H: human-human protein interaction (blue edges). V-H_{HCV} represents the interactions between HCV and human proteins (black box). H_{HCV}-H_{HCV} is composed of human proteins interacting with viral proteins (red box). H-H network represents interactions between human proteins (blue box).

B. Number of proteins and interactions in HCV-human interaction network. Number of human proteins interacting with HCV proteins (H_{HCV}) and corresponding number of protein-protein interactions (V-H_{HCV} PPI). Data are given for our yeast two-hybrid screens (IMAP Y2H) and for literature curated interactions (IMAP LCI).

C. Validation of Y2H interactions by co-affinity purification assay. Nine co-AP positive assays are shown, representing: NS5A-SORBS2 (1), NS3-CALCOCO2 (2), NS5A-BIN1 (3), NS5A-MOBK1B (4), NS5A-EFEMP1 (5), NS3-PSMB9 and NS5A-PSMB9 (6), NS5A-PPPIRI3L (7), NS3-RASAL2 (8). After pull-down with GST tagged viral baits (+) or with negative control GST alone (−), cellular preys are identified with anti-Flag antibody. Anti-GST antibody identifies either GST alone or GST-tagged viral baits. Expression of cellular preys in cell lysate is controlled by anti-Flag (bottom panel).

FIG. 2: Graphical Representation of the HCV-Human Interaction Network.

A. Graphical representation of H-H network. Each node represents a protein and each edge an interaction. Red and blue nodes are respectively H_{HCV} and H_{NOT-HCV}.

B. Graphical representation of V-H_{HCV} interaction network. Black node: viral protein; Red node: human protein; Red edge: interaction between human and viral proteins (V-H_{HCV}); Blue edge: interaction between human proteins (H_{HCV}-H_{HCV}). The largest component containing 196 proteins is represented in the middle of the network. Names of cellular proteins belonging to the three other connected components are also represented.

FIG. 3: Topological Analysis of the HCV-Human Interaction Network.

A. Topological analysis of H and H_{HCV} in H-H network. Degree (k), betweenness (b) and shortest path (l) were computed for all human proteins and for H_{HCV} from the IMAP Y2H dataset.

B. Degree and Betweenness Distribution of H and H_{HCV} Proteins in H-H Network. Normalised log degree (left) and log betweenness (right) distribution of H (blue) and

H_{HCV} proteins (red). Solid line represents linear regression fit. Vertical dashed lines give mean degree and betweenness values. Each class is represented with conventional standard error.

C. Degree and betweenness correlation of H in H-H network. Normalised log degree (x axis) and log betweenness (y axis) of H proteins into H-H network. Black solid line represents the linear regression fit (R²=0.56). Horizontal and vertical dashed lines give respectively the mean degree and betweenness values. Low degree (LD) and high degree (HD) classes were defined by using the average degree cut-off.

D. Mean degree and betweenness of H_{NOT-HCV} and H_{HCV} for low and high degree proteins. Top: mean betweenness (log scale) of H_{NOT-HCV} (blue) and H_{HCV} (red) is given for LD and HD classes. Bottom: mean degree of H_{NOT-HCV} (blue) and H_{HCV} (red) is given for LD and HD classes. The conventional standard error threshold and the U test p-value are represented (***: p-value<10⁻¹⁰, NS: not significant).

FIG. 4: IJT Network

A. Graphical representation of IJT network. Proteins (nodes) members of insulin (blue), Jak/STAT (red) and TGFβ (green) pathways according to KEGG annotation, and their interactions (edges) are shown (proteins interacting with HCV proteins are named). Proteins shared by two pathways are shown in secondary colours (pink, yellow and cyan). Grey and black nodes are neighbours that connect the KEGG pathways and that interact with HCV proteins (grey: protein from the IMAP Y2H dataset, black: protein from IMAP LCI dataset). Neighbours interacting with HCV but not connecting the KEGG pathways are not represented. Discussed protein examples PLSCR1 and YY1 are in box. Interactive visualization tools are provided in supplementary files (Network visualization).

B and C. Relative contribution of each viral protein in V-H_{HCV} and IJT network. Percentage of the three most interacting viral proteins is given. 51.3% of CORE interactions are concentrated in the IJT network.

FIG. 5: Interaction of HCV with Focal Adhesion

A. Schematic representation of focal adhesion adapted from KEGG (ID: Hs04510). H_{HCV} are represented by orange boxes and H_{NOT-HCV} by blue boxes.

B and C. Functional validation of focal adhesion perturbation by NS3 and NS5A.

96-well plates were coated with fibronectin (B) or poly-L-lysine (C) at various concentrations. 293T cells expressing NS2, NS3, NS3/4A or NS5A were plated on the matrix for 30 min. Adherent cells were stained with crystal violet. FA50 is the matrix concentration necessary for half maximum adhesion. Values represent mean of three independent experiments with their standard deviation.

FIG. 6: The HCV ORFeome.

Schematic representation of the HCV genome and definition of ORFs designed for Y2H screens. The HCV positive strand genome (purple) encodes a polyprotein (orange) which is co-translationally processed in 10 proteins. Red: full length protein; blue: domain; yellow+green: NS4A+NS3 chimeric fusion; pink: NS5A membrane anchor. Genome and polyprotein coordinates of each construction are given in the table.

FIG. 7: Degree and Betweenness Distributions of H, H_{HCV} and H_{EBV} Proteins in H-H Network.

log degree (left) and log betweenness (right) distributions of H proteins (blue), H_{HCV} (red) and H_{EBV} (green). Solid lines represent linear regression fits. Vertical dashed lines give mean degree and betweenness values.

### TABLE S1: H_{HCV} LISTING

HCV proteins are referenced according to their NCBI mature peptide product name (column 1). Human proteins are referenced with their cognate NCBI gene name and gene ID (columns 2 and 3). The number of IST for IMAP Y2H (IMAP1 and IMAP2, according to the method of screening) is given in columns 4 and 5. IMAP LCI (Literature Curated Interactions) from text-mining and BIND database associated PubMed IDs are given in columns 6 and 7. Co-affinity purification (CoAP) or Y2H pairwise matrices validations are indicated in columns 8 and 9 (+: IMAP validation, −: not validated, NA: non assayable due to default of protein expression or to cellular protein directly interacting with GST).

### TABLE S2: LISTING OF HUMAN CELLULAR PROTEINS INTERACTING WITH MORE THAN ONE VIRAL PROTEIN

Human proteins are referenced with their cognate NCBI gene name (column 1). HCV proteins are referenced according to their NCBI mature peptide product name (column 2). Origin of the dataset (IMAP Y2H, IMAP LCI, column 3).

### TABLE S3: TOPOLOGICAL ANALYIS OF THE HCV-HUMAN NETWORK

### Connected Components of the HCV-Human Network.

The size of the largest component and the number of connected components of V-H_{HCV} sub-network were computed (IMAP dataset, column 2). In order to test the significance of observed values, we computed the mean of the largest component size and the mean number of connected components obtained after 1000 simulations of random sub-networks (IMAP Sim column 3). The differences between the observed and the simulated values were highly significant (***: p-value<10⁻¹⁰).

### Topological Properties of the H_{HCV} and H_{EBV} Proteins in the Human Interactome.

Full interactome (A), high-confidence interactome (B, containing only PPIs with at least two PMIDs or validated by two different methods). The number of proteins and PPIs that can be integrated into the human interactome are given for H_{HCV} and H_{EBV}. Percentage of H_{HCV} and H_{EBV} that are present in the human interactome are given according to the origin of the dataset. Average degree (k), betweenness (b) and shortest path (l) were computed for H_{HCV} and H_{EBV} in both full and high-confidence interactomes (25).

### TABLE S4: KEGG PATHWAY ENRICHMENT FOR H_{HCV}

Over-represented KEGG pathways were identified as significant after multiple testing adjustments (adjusted p-value<5.10⁻²) and are listed by viral protein. For each pathway, number of H_{HCV} is given, with the relative contribution of IMAP Y2H dataset between brackets. Black boxes highlight discussed pathways.

### TABLE S5: HCV PROTEIN DISTRIBUTION AND ENRICHMENT IN IJT NETWORK

A. H_{HCV} enrichment in IJT network for each viral protein. Number of H_{HCV} is given in V-H_{HCV} and IJT networks. Enrichment of H_{HCV} in IJT network was tested with exact Fisher test for each viral protein. Associated odd ratios and p-values are given.

B. H_{HCV} enrichment in Jak/STAT, TGFβ and Insulin pathways for each viral protein. Number of H_{HCV} is given in V-H_{HCV} network and Jak/STAT, TGFβ and Insulin pathways (as defined in KEGG database). Enrichment of H_{HCV} in Jak/STAT, TGFβ and Insulin pathways was tested with exact Fisher test for each viral protein. Associated odd ratios and p-values are given.

C. H_{HCV} enrichment in Jak/STAT, TGFβ and Insulin inter-pathways for each viral protein. Inter-pathways are defined as the H_{HCV} connecting two or three KEGG pathways. Number of H_{HCV} is given in V-H_{HCV} network and Jak/STAT, TGFβ and Insulin inter-pathways. Enrichment of H_{HCV} in Jak/STAT, TGFβ and Insulin inter-pathways was tested with exact Fisher test for each viral protein. Associated odd ratios and p-values are given.

### Example 1

### Summary

A proteome-wide mapping of interactions between hepatitis C virus and human proteins was performed to provide a comprehensive view of the cellular infection. A total of 314 protein-protein interactions between HCV and human proteins was identified by yeast two-hybrid and 170 by literature mining. Integration of this dataset into a reconstructed human interactome showed that cellular proteins interacting with HCV are enriched in highly central and interconnected proteins. A global analysis based on functional annotation highlighted the enrichment of cellular pathways targeted by HCV. A network of proteins associated with frequent clinical disorders of chronically infected patients was constructed by connecting the insulin, Jak/STAT and TGFβ pathways with cellular proteins targeted by HCV. CORE protein appeared as a major perturbator of this network. Focal adhesion was identified as a new function affected by HCV, mainly by NS3 and NS5A proteins.

### Material & Methods:

### Construction of the HCV ORFeome.

HCV genome is a positive strand RNA molecule, encoding one polyprotein which is cleaved by cellular and viral proteases in structural proteins (CORE, E1, E2 and p7), and non-structural proteins (NS2, NS3, NS4A, NS4B, NS5A and NS5B) (1). All proteins were cloned in full length and domains except for NS4B for which no domain has been designed, using the euHCVdb facilities (http://www.euhcvdb.ibcp.fr (2)) (FIG. 6). Fusion NS4A-NS3 protein, as well as NS4A-NS3 protease domain were constructed (3, 4). All 27 ORFs from the HCV genotype 1b, isolate con1 (AJ238799) (5), were cloned in a Gateway recombinational cloning system. Each ORF was PCR amplified (with KOD polymerase, Novagen) using attB1.1 and attB2.1 recombination sites fused to forward and reverse primers, then cloned into pDONR223 (6). All entry clones were sequence verified.

### Yeast Two Hybrid (Y2H) Library Screens.

HCV ORFs were transferred from pDONR223 into bait vector (pPC97) to be expressed as Gal4-DB fusions in yeast. Two different screening methods were used (IMAP1 and IMAP2). Both for IMAP1 and IMAP2 strategies and because bait constructs sometimes self-transactivate reporter genes, SD-L-H culture medium were supplemented with 3-aminotriazole (3-AT). Appropriate concentrations of this drug were determined by growing bait strains on SD-L-H medium supplemented with increasing concentrations of 3-AT. Self-transactivation by NS5A without its membrane anchor was too high to be titrated with 3-AT and was of further tested. For IMAP1, bait vectors were introduced in MAV203 yeast strain and both human spleen and foetal brain AD-cDNA libraries (Invitrogen) were screened by transformation as described (7). All primary positive clones (selected on SD-W-L-H+3-AT) were tested by further phenotypic assay using two additional reporter genes: LacZ (X-Gal colorimetric assay) and URA3 (growth assay on 5-FOA supplemented medium). Positive clones that displayed at least 2 out of 3 positive phenotypes were retested into fresh yeasts. Clones that did not retest were discarded. AD-cDNA were PCR-amplified and inserts were sequenced to identify interactors. IMAP2 screens were performed by yeast mating, using AH109 and Y187 yeast strains (Clontech (8)). Bait vectors were transformed into AH109 (bait strain) and human spleen and foetal brain AD-cDNA libraries (Invitrogen) were transformed into Y187 (prey strain). Single bait strains were mated with prey strains then diploids were plated on SD-W-L-H+3-AT medium. Positive clones were maintained onto this selective medium for 15 days to eliminate any contaminant AD-cDNA plasmid (9). AD-cDNAs were PCR amplified and inserts were sequenced.

### IST (Interactor Sequence Tag) Analysis

We have developed a bioinformatic pipeline that assigns each IST to its native human genome transcript. First, ISTs were filtered by using PHRED (10, 11) at a quality score superior to the conventional 20 threshold value (less than 1% sequence errors). Gal4 motif was searched (last 87 bases of GAL4-AD), sequences downstream of this motif were translated into peptides and aligned using BLASTP against the REFSEQ (http://www.ncbi.nlm.nih.gov/RefSeq/) human protein sequence database (release 04/2007). Low-confidence alignments (E value>10⁻¹⁰, identity <80%) and premature STOP codon containing sequences were eliminated. Only in-frame proteins and high quality sequences were further considered

### Integrated Human Interactome Network (H-H Network)

Only physical and direct binary protein-protein interactions were retrieved from BIND (12), BioGRID (13), DIP (14), GeneRIF (15), HPRD (16), IntAct (17), MINT (18), and Reactome (19). NCBI official gene names were used to unify protein ACC, protein ID, gene name, symbol or alias defined in different genome reference databases (i.e ENSEMBL, UNIPROT, NCBI, INTACT, HPRD . . . ) and to eliminate interaction redundancy due to the existence of different protein isoforms for a single gene. Thus, the gene name was used in the text to identify the proteins. Finally, only non-redundant protein-protein interactions were retained for building the human interactome dataset, i.e if A interacts with B and B with A, only A with B interaction was selected.

### Text-Mining of Interactions Between HCV and Cellular Proteins

Literature curated interactions (LCI), describing binary interactions between cellular and HCV proteins, were extracted from BIND database and PubMed (publications before August 2007) by using an automatic text-mining pipeline completed by expert curation process. For the text-mining approach, all abstracts related to “HCV” and “protein interactions” keywords were retrieved, subjected to a sentencizer (sentence partition) and a part-of-speech tagger for gene name (based on NCBI gene name and aliases) and interaction verbs (interact, bind, attach . . . ) (20). Sentences presenting co-occurrences of at least one human gene name, one viral gene name and one interaction term, were prioritized to curation by human expert.

### Validation by Co-Affinity Purification

A random pool of 59 IMAP Y2H interactions was chosen for CoAP assays. Cellular ORFs (interacting domains found in Y2H screens) were cloned by recombinational cloning from a pool of human cDNA library or the MGC cDNA plasmids using KOD polymerase (Toyobo) into pDONR207 (Invitrogen). After validation by sequencing, these ORFs were transferred to pCi-neo-3xFLAG gateway-converted, and HCV ORFs were transferred into pDEST27 (GST fusion in N-term). HEK-293T cells were then co-transfected (JetPei, Polyplus) by each pair of plasmid encoding interacting proteins. Controls are GST alone against 3xFLAG-tagged preys. Two days after transfection, cells were harvested and lysed (0.5% NP-40, 20 mM Tris-HCl (pH 8.0), 180 mM NaCl, 1 mM EDTA, and complete protease inhibitor cocktail). Cell lysates were cleared by centrifugation for 20 min at 13,000 rpm at 4° C. and soluble protein complexes were purified using Glutathione Sepharose 4B beads (GE Healthcare). Beads were then washed extensively four times with lysis buffer and proteins were separated on SDS-PAGE and transferred to nitrocellulose membrane. GST-tagged viral proteins and 3xFLAG-tagged cellular proteins were detected using standard immunoblotting techniques using anti-GST (Covance) and anti-FLAG M2 (Sigma) monoclonal antibodies (21).

### Network Visualization

Large Graph Layout (http://bioinformatics.icmb.utexas.edu/lgl/) was applied to visualize the H-H network in FIG. 2A. Guess tool (http://graphexploration.cond.org/) was used to graphically represent H-H_{HCV} infection network in FIG. 2B and the IJT network in FIG. 4A. FIGS. 2B and 4A are available in a GUESS interactive format (GUESS Data Format) in SF1.tar.gz (infection_network.properties and ijt_network.properties).

### Topological Analysis

The R (http://www.r-project.org/) statistical environment was used to perform statistical analysis and the igraph R package (http://cneurocvs.rmki.kfki.hu/igraph/) to compute network connected components, centrality (degree, betweenness) and shortest path measures.

### Connected Component:

In an undirected network, a connected component is a maximal connected sub-network. Two nodes are in the same connected component if and only if there exist a path between them. We also included in connected components proteins that are not connected to any other protein, according to igraph R package.

### Degree:

The degree of a node (k) is the number of edges incident to the node. The mean degree of human proteins was computed and was compared to the mean degree of all H_{HCV}.

### Shortest Path:

The shortest path problem is the finding of a path between two nodes such that the sum of the weights of its constituent edges is minimized. The shortest paths (l, also called geodesics) are calculated here by using breath-first search in the graph. Edge weights are not used here, i.e every edge weight is one. The mean shortest path between any two pairs of human proteins was computed and was compared to the mean shortest path between any two pairs H_{HCV}.

### Betweenness:

The node betweenness (b) are roughly defined by the number of shortest paths going through a node. The mean betweenness of all human proteins was computed and compared to the mean shortest path between any two pairs of H_{HCV}.

### Topological Analysis Statistical Test:

The Wilcoxon Mann-Withney rank sum test (the U test) was chosen to statistically challenge observed differences. The U test is a non-parametric alternative to the paired Student's t-test for the case of two related samples or repeated measurements on a single sample. The generalized linear model and ANOVA analysis was used to respectively model and test the separate and additive effects of degree and betweenness on the probability that HCV proteins interact with human proteins.

### Functional Analysis Using KEGG Annotations

Cellular pathway data were retrieved from KEGG (22), the Kyoto Encyclopedia of Genes and Genomes (http://www.genome.jp/kegg/) and were used to annotate NCBI gene functions. For each viral-host protein interactors, the enrichment of specific KEGG pathway was tested by using an exact Fisher test (pvalue<5 10⁻²) followed by the Benjamini and Hochberg multiple test correction (23) in order to control false discovery rate.

### Cell-Adhesion Assay

Serial dilutions (from 10 to 0.04 μg/ml) of fibronectin or poly-L-lysine in PBS were coated on 96-well microtiter plates overnight at 4° C. Non-specific binding sites were saturated at room temperature with PBS 1% BSA for 1 h. HEK 293T cells were transfected with pCineo3xFlag NS2, NS3, NS3/4A or NS5A (JetPei, Polyplus), collected 2 days later with 2 mM EDTA in PBS, spread in triplicate at 1.10⁵ cell/well in serum-free medium with 0.1% BSA, and incubated for 30 min at 37° C. Non-adherent cells were washed away and adherent cells were fixed with 3.7% paraformaldehyde. Cells were stained with 0.5% crystal violet in 20% methanol for 20 min at room temperature and washed 5 times in H₂O. Staining was extracted 50% ethanol in 50 mM sodium citrate, pH4.5, and the absorbance was read at 590 nm on an ELISA reader (MRX microplate reader, Dynatech Laboratories). Values were normalized to 100% adhesion at 10 μg/ml. The percentage of adhesion was determined for each cell type at each matrix concentration. 50% of maximum adhesions (FA50) were calculated from the curves (Adapted from Miao et al (24)).

### REFERENCES CITED IN MATERIAL & METHODS

1. F. Penin, J. Dubuisson, F. A. Rey, D. Moradpour, J. M. Pawlotsky, Hepatology 39, 5 (2004).
2. C. Combet et al., Nucleic Acids Res. 35, D363 (2007).
3. J. L. Kim et al., Cell 87, 343 (1996).
4. S. S. Taremi et al., Protein Sci. 7, 2143 (1998).
5. V. Lohmann et al., Science 285, 110 (1999).
6. J. F. Rual et al., Genome Res. 14, 2128 (2004).
7. S. Li et al., Science 303, 540 (2004).
8. M. Albers et al., Mol. Cell. Proteomics 4, 205 (2005).
9. P. O. Vidalain, M. Boxem, H. Ge, S. Li, M. Vidal, Methods 32, 363 (2004).
10. B. Ewing, P. Green, Genome Res. 8, 186 (1998).
11. B. Ewing, L. Hillier, M. C. Wendl, P. Green, Genome Res. 8, 175 (1998).
12. G. D. Bader, D. Betel, C. W. Hogue, Nucleic Acids Res. 31, 248 (2003).
13. C. Stark et al., Nucleic Acids Res. 34, D535 (2006).
14. I. Xenarios et al., Nucleic Acids Res. 30, 303 (2002).
15. Z. Lu, K. B. Cohen, L. Hunter, Pac. Symp. Biocomput., 269 (2007).
16. S. Peri et al., Nucleic Acids Res. 32, D497 (2004).
17. S. Kerrien et al., Nucleic Acids Res. 35, D561 (2007).
18. A. Chatr-aryamontri et al., Nucleic Acids Res. 35, D572 (2007).
19. I. Vastrik et al., Genome Biol. 8, R39 (2007).
20. D. Rebholz-Schuhmann, M. Arregui, S. Gaudan, H. Kirsch, A. Jimeno, Bioinformatics (2007).
21. J. F. Rual et al., Nature 437, 1173 (2005).
22. K. F. Aoki-Kinoshita, M. Kanehisa, Methods Mol. Biol. 396, 71 (2007).
23. Y. Benjamini, D. Drai, G. Elmer, N. Kafkafi, I. Golani, Behav. Brain Res. 125, 279 (2001).
24. H. Miao, E. Burnett, M. Kinch, E. Simon, B. Wang, Nat. Cell. Biol. 2, 62 (2000).
25. M. A. Calderwood et al., Proc. Natl. Acad. Sci. U.S.A. 104, 7606 (2007).

### Results

### Construction of a HCV-Human Interactome Map.

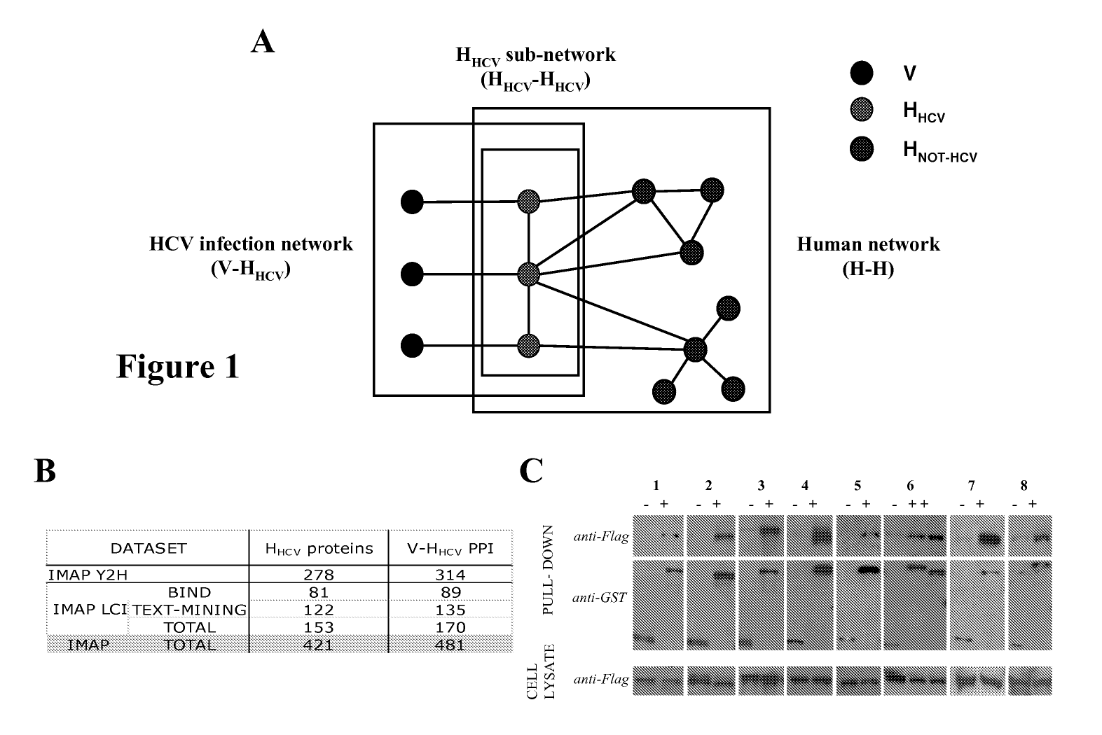

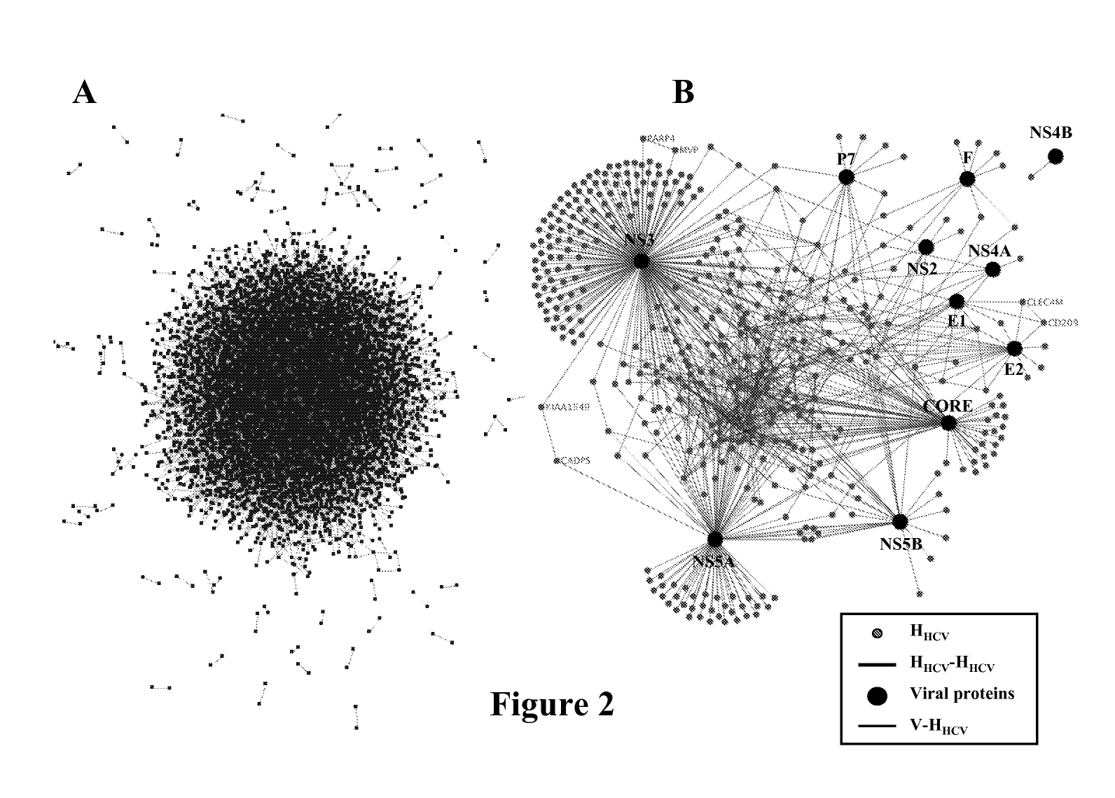

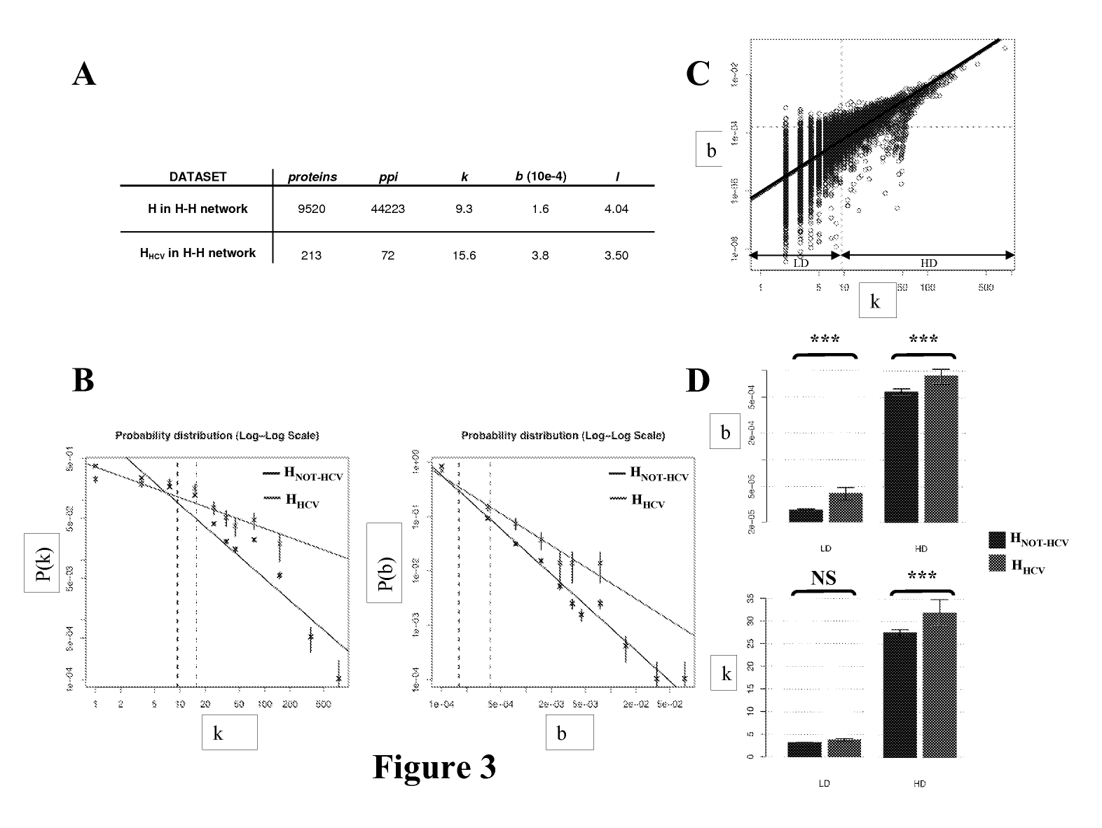

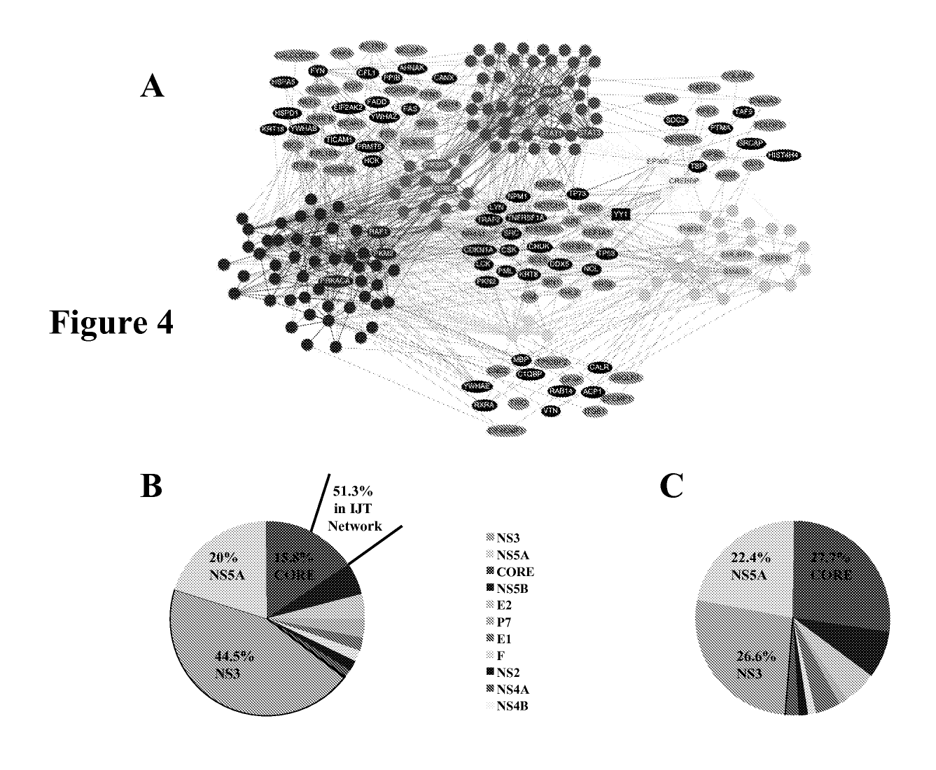

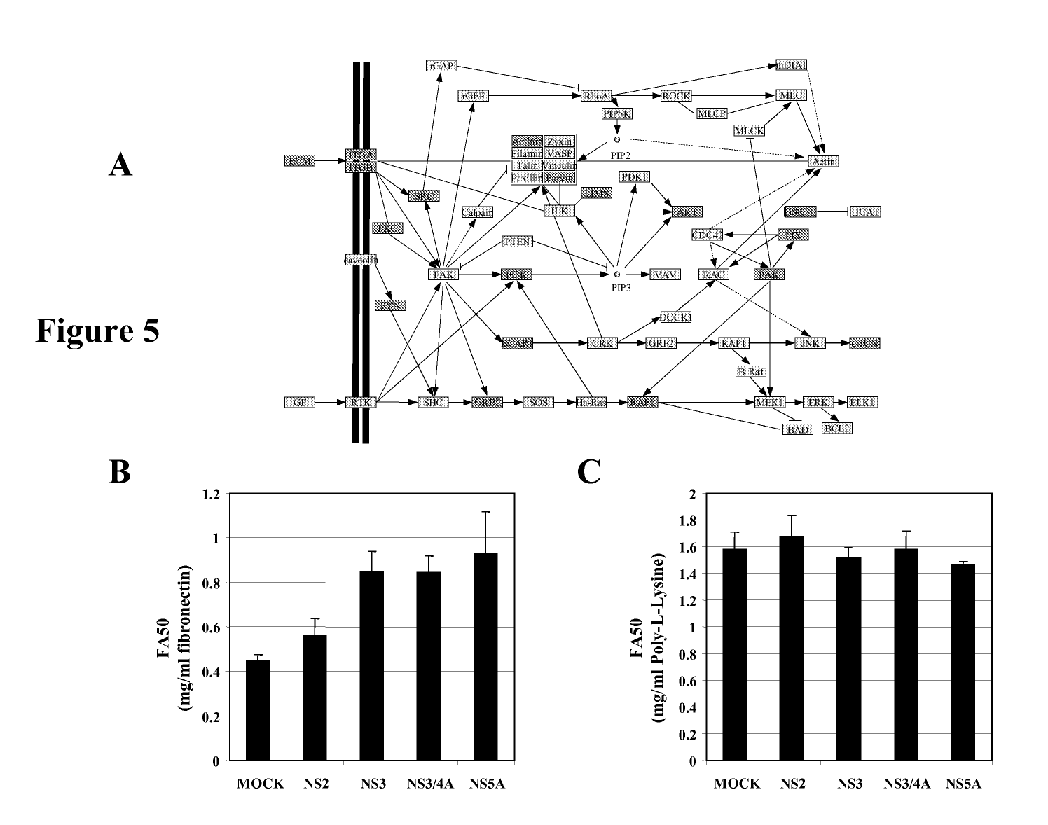

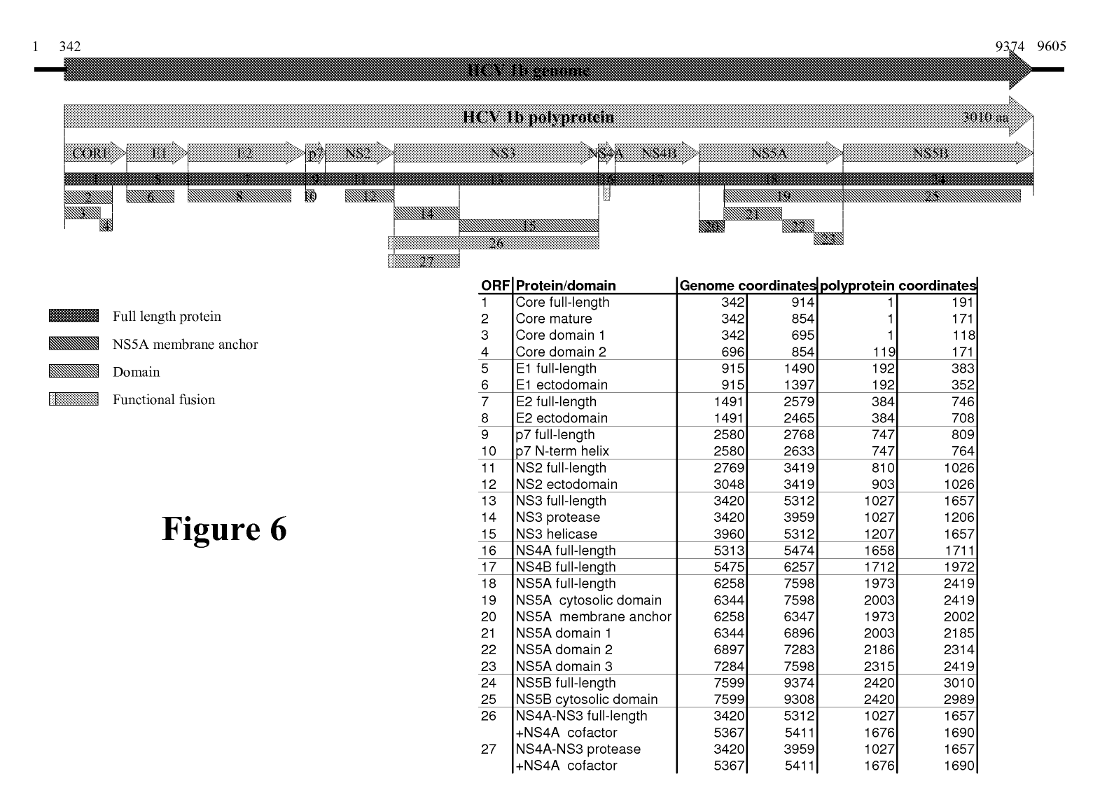

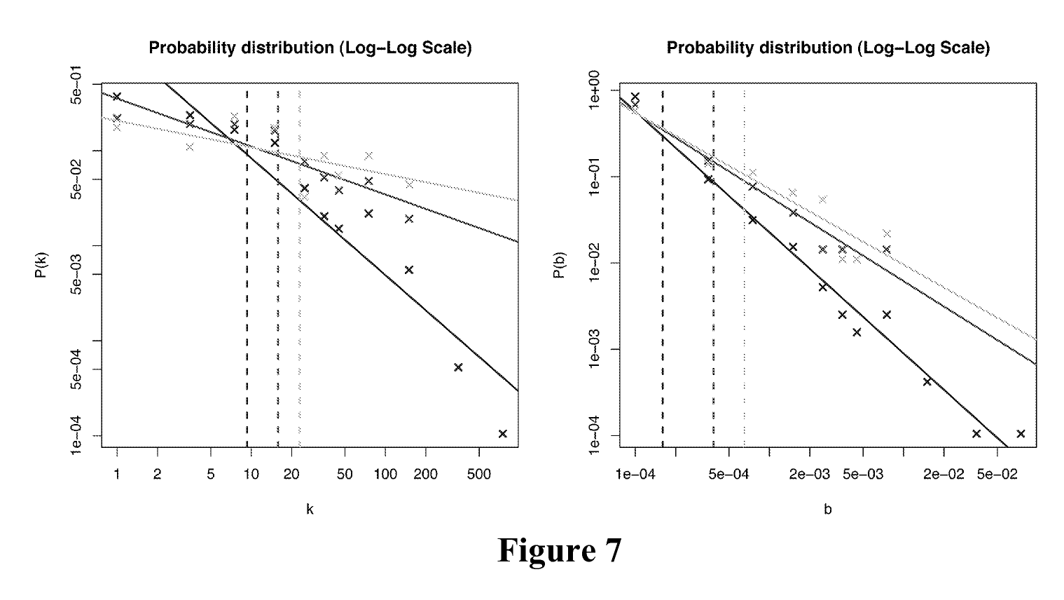

A comprehensive interactome map between HCV and cellular proteins was generated by Y2H screens. Twenty seven constructs encoding full-length HCV mature proteins or discrete domains were cloned using a recombination-based cloning system (10) (FIG. 6). Four independent screens were performed with each HCV bait protein, probing two distinct human cDNA libraries (Supplementary Methods). 314 HCV-human PPIs were identified, involving 278 human proteins (FIG. 1B, IMAP Y2H dataset in table S1). Pairwise interactions between HCV and human proteins were also extracted from the literature by automatic text mining and checked by expert curation (Supplementary Methods, IMAP LCI dataset in FIG. 6). 135 PPI were extracted from Pubmed and 89 from BIND database (11) (FIG. 1B). The resulting HCV-human interactome is thus composed of 481 PPI with 65% new interactions, involving 11 HCV proteins and 421 distinct human proteins (FIG. 1B). The low redundancy between IMAP Y2H and IMAP LCI datasets emphasizes a high false-negative rate of the Y2H system which is in agreement with recent studies (12, 13). Two validation methods were used to assess the confidence of the IMAP Y2H dataset. Two third of the dataset was validated by Y2H pairwise matrices. From the remaining interactions, 25% were randomly selected and tested by co-affinity purification giving rise to a validation rate over 85% (FIG. 1C and table S1). This Y2H dataset was thus of very high confidence for further analysis at the topological and functional level. Analysis of the HCV-infection network (V-H_{HCV}, FIG. 1A) showed that NS3, NS5A and CORE are the most connected proteins in the human interactome, with 214, 96 and 76 cellular partners respectively, highlighting the potential multi-functionality of these proteins during infection (Table S1, FIG. 4B). In addition 45 cellular proteins are targeted by more than one viral protein, suggesting their essentiality for virus biology (7) (Table S2).

A human PPI network (H-H network, FIG. 1A) was reconstructed from 8 databases (14) (Supplementary Methods). This network is composed of 44,223 non-redundant PPI between 9,520 different proteins (FIG. 2A). Interestingly, whereas only 30% of human proteins are present in this dataset, human proteins targeted by HCV (H_{HCV}) are clearly over-represented in this H-H network (IMAP Y2H dataset: 76% and IMAP LCI dataset: 92%, exact Fisher test p-value<2.2 10⁻¹⁶). This suggests that HCV preferentially targets host proteins already known to be engaged in protein-protein interactions (12, 15). For the IMAP LCI dataset, the higher percentage of H_{HCV} integrated in the human interactome may be explained by inspection bias of well-studied proteins and biological pathways. Analysis of H_{HCV}-H_{HCV} sub-network showed that cellular proteins interacting with HCV are significantly more interconnected than expected for random sub-networks (FIG. 1A, 2B, Supplementary Methods). Indeed, the 338 H_{HCV} integrated into the human interactome are distributed into 131 connected components (versus 276 expected by random sub-networks p-value<10⁻¹⁰, Table S3). The largest one is composed of 196 H_{HCV} (versus 18 expected by random sub-networks p-value<10⁻¹⁰) in contrast to 127 components containing only one protein. The three remaining connected components comprised two proteins. Two contained functionally related proteins (CLEC4M and CD209 are lectins involved in viral entry (16); MVP and PARP4 are involved in Vault complex (17)) and one contained proteins not known to be functionally linked (KIAA1549 and CADPS).

### Topological Analysis of the HCV-Human Interaction Network

To assess how HCV proteins interplay with the cellular protein network, we next focused on the centrality measures of H_{HCV} proteins integrated into the H-H interactome. Local (degree) and global (shortest path and betweenness) centrality measures were calculated. Briefly, the degree (k) of a protein in a network corresponds to its number of direct partners and is therefore a measure of local centrality. Betweenness (b) is a global measure of centrality as it measures the number of shortest paths (the minimum distance between two proteins in the network, l) that pass through a given protein. The average degree, betweenness and shortest path of the H-H network are respectively 9.3, 1.6 10⁻⁴ and 4.04, which is in good agreement with previous reports (18) (FIG. 3A). In order to provide an unbiased analysis, calculations were based on the 213 H_{HCV} from the IMAP Y2H dataset integrated in the human interactome. The average degree of H_{HCV} is significantly higher than average degree of the human interactome (15.6 versus 9.3, U test p-value<10⁻³). The comparison of degree probability distribution reveals that H_{HCV} are preferentially represented in all class above the mean degree (FIG. 3B, left). This indicates that HCV proteins have a strong tendency to interact with highly connected cellular proteins. However, as degree measures only local connectivity of proteins, global characteristics that could reflect information exchange and propagation in the network were investigated (19). At a global scale, the average betweenness of H_{HCV} was significantly higher than the average betweenness of the human interactome (3.8 10⁻⁴ versus 1.6 10⁻⁴, U test p-value<10⁻³). As for the degree, the comparison of betweenness probability distribution shows an excess of H_{HCV} in all class above the mean betweenness (FIG. 3B, right). In addition, the lower average shortest path found between H_{HCV} proteins compared to the average shortest path in the H-H network reveals the topological vicinity of H_{HCV} (3.50 versus 4.04, U test pvalue<10⁻⁵). Both local and global centrality of H_{HCV} from the IMAP LCI dataset were higher than for the IMAP Y2H dataset, emphasizing the problem of literature inspection bias and reinforcing the unbiased approach of Y2H screening (Table S3). To ensure that the preferential attachment to central H_{HCV} was not due to inherent bias in the H-H interactome, we performed the same analysis with a highly confident, but less comprehensive human interactome (Table S3, Supplementary methods). This trend was maintained with this dataset, confirming that H_{HCV} are highly central within the human interactome, both locally and globally, and appear relatively close to each other in this network. For comparative analysis of HCV and EBV, the centrality measures were also computed for H_{EBV} (dataset from Calderwood et al. (7)). Degree, betweenness and shortest path followed the same tendency with H_{EBV} proteins (Table S3 and FIG. 7) and were in good agreement with a previous report (7). These results indicate that preferential attachment on central proteins may be a general hallmark of viral proteins as recently suggested by analysis of the literature (9). The high centrality of these proteins was previously shown to correlate with their functional essentiality for the cell (20). More precisely, lethal and disease related proteins were found enriched in central proteins (19, 21-23).

In order to determine which of the degree or the betweenness most influences the probability of interaction between viral and cellular proteins, we used a generalized linear model to test the separate and additive effects of both measures (Supplementary Methods). This analysis revealed that betweenness better explain the probability of interaction between viral and human proteins (ANOVA p<10⁻³). FIG. 3C shows a partial correlation between k and b centrality measures (R²=56%, p-value<10⁻¹⁶), explained by the high variability of betweenness at low degree values. We thus asked whether this high variability observed at low degree could explain the preponderant effect of betweenness. For this purpose, the datasets were split in low (LD) and high degree (HD) protein classes according to the average degree of the human interactome. For cellular proteins included in LD class, HCV interact preferentially with proteins of high-betweenness independently of their degree property (FIG. 3D). Within the HD class, interaction with HCV proteins is dependent on both betwenness and degree of cellular proteins. Based on a recent study in yeast (24), it can be extrapolated that low-degree high-betweenness H_{HCV} proteins could act as connectors or bottlenecks between cellular modules and may thus be essential for the infection.

### Functional Analysis of the HCV-Human Interaction Network

In order to better understand biological functions targeted by HCV, we next tested the enrichment of specific pathways for all interactors of a given viral protein. This was done by analyzing the H_{HCV} proteins in regards to the KEGG functional annotation pathways (Table S4, Supplementary Methods). Although this approach is not totally unbiased because functions have not yet been attributed to all proteins, it remains a powerful way of incorporating conventional biology in system-level datasets. This analysis showed an enrichment for three pathways associated with HCV clinical syndromes (insulin, TGFβ and Jak/STAT pathways) and identified focal adhesion as a novel pathway affected by HCV.

Chronic infection by HCV is associated with an increased risk for metabolic disorders with development of steatosis. Insulin resistance is a common feature of this process. It also contributes to liver fibrosis and is a predictor of a poor response to interferon-α (IFN-α) anti-viral therapy (25, 26). Conversely, IFN-α can prevent fibrosis progression (27). TGFβ plays a crucial role in maintaining cell growth and differentiation in the liver. It is a strong profibrogenic cytokine whose production is frequently enhanced during infection. Impaired TGFβ response is also observed during HCV infection (28). Although insulin, TGFβ and Jak/STAT pathways have been suspected to be involved in these clinical features (29), their closely related perturbation during HCV infection remain largely unexplained. We thus used a network approach to identify cellular proteins targeted by HCV and localized at the interface of these pathways. The resulting interaction map was constructed to form the IJT network (Insulin-Jak/STAT-TGFβ, FIG. 4A). Sixty-six H_{HCV} proteins are connecting two pathways while 30 H_{HCV} proteins are connecting the three pathways. Interaction of these proteins with HCV proteins may thus induce functional perturbations that could expand to adjacent pathways. One of these proteins is PLSCR1 (Scramblase 1), connecting insulin and Jak/STAT pathways. Known to be involved in redistribution of plasma membrane phospholipids (30), this protein is also a potential activator of genes in response to interferon and its knock-down with siRNA favours viral replication (31). Interestingly, pLSCR1⁻/⁻ mice also exhibit an onset of insulin resistance (32). Although not annotated in the Insulin or Jak/STAT pathways, PLSCR1 thus appears essential for the functionality of these pathways. By interacting with PLSCR1, CORE could therefore interfere with both Jak/STAT and insulin pathways. Another example is the nuclear factor Yin Yang 1 (YY1) which exhibits a more central position in the IJT network as it connects the three pathways. HCV CORE interaction with YY1 has been previously shown to be functional relieving NPM1 expression. This observation could be extrapolated to PPARδ expression and SMADs transcriptional activity in support of insulin and TGFβ pathway modulation (33-35). These are only two illustrative examples of cellular targets likely to be involved in HCV-induced phenotypes. These examples bring us back to the logical reductionist approach of hepatitis C in an effort to provide better information about molecular mechanisms correlating to clinical syndromes. Although this molecular approach of the pathology is applicable to basal element of a system (proteins in this work) some of the clinical phenotypes observed in chronic HCV infection are likely to result from the integrative effect of protein interactions depicted in the IJT network. In addition, the reductionist approach cannot always be applicable at a system level because the robustness property of a network can confer its ability to remain functional in face of different perturbations.

Another issue that became apparent in the IJT network is that CORE protein mediates proportionally more interactions than the other HCV proteins (FIG. 4B, 4C). Indeed, preferential interaction with IJT network was only observed with CORE (51.3%, Table S5). As a consequence, CORE makes 27.7% of the interactions in the IJT network, corresponding to a significant enrichment (exact Fisher test p-value<10⁻⁴). More precisely, this protein is over-represented in Jak-STAT and TGFβ pathways (exact Fisher test p-value<0.05) and in H_{HCV} connecting Insulin/Jak/STAT and Insulin/TGFβ pathways (exact Fisher test p-value<0.05, Table S5). CORE thus appears as a major perturbator of the IJT network. Interestingly, transgenic mice expressing CORE develop insulin resistance (36, 37). A proposed mechanism was that CORE-induced SOCS3 promotes proteasomal degradation of IRS1 and IRS2 through ubiquitination (38). As SOCS3 is also a negative regulator of Jak/STAT pathway, this could explain the occurrence of IFN-α resistance. Clearly, the IJT network indicates that the action of CORE is likely to be much more complex that previously thought. Although the IJT network can not yet be analyzed dynamically, it remains that it provides a unique way of deciphering some of the complex disorders associated with chronicity. It is also worth considering that the IJT network may identify a series of genes involved in diseases, such as steatosis and fibrogenesis, in the absence of viral infection.

Focal adhesion was over-represented as a new function targeted by NS3 and NS5A proteins, with a major contribution of data generated by IMAP Y2H screens (Table S4). Integrin-linked focal adhesion complexes control cell adhesion to extracellular matrix (ECM) and association of these complexes with actin-cytoskeleton plays a major role in cell migration. Upon binding to the ECM, both α and β integrin subunits recruit proteins establishing a physical link between the actin-cytoskeleton and signal transduction pathways. When deregulated, this functional process can lead to perturbation of cell mobility, detachment from the ECM and tumour initiation and progression. FIG. 5A shows KEGG focal adhesion pathway with proteins targeted by HCV, mainly NS3 and NS5A proteins. Impact of single expression of NS3, NS3/4A or NS5A on focal adhesion functionality was assessed using a cellular adhesion assay on fibronectin and poly-L-lysine. These viral proteins inhibited cell adhesion to fibronectin compared to MOCK or NS2 expressing cells (FIG. 5B). By contrast adhesion to poly-L-lysine, which does not engage integrins, was not affected (FIG. 5C). The same inhibition level was observed for NS3/4A and NS3 suggesting that the enzyme activity of this protease does not have a major effect on focal adhesion perturbation. In addition to initiation and progression of cancer, the engagement of focal adhesion by HCV could have consequences on viral spreading. Interference with several steps of the actin-cytoskeleton remodelling has been described for retroviruses which can exploit this process to surf along cellular protrusions of target cells to reach the entry site (39). It is conceivable that a related process, involving binding of the viral envelop to integrins, could be exploited by HCV to favour its transmission. In a network approach of HCV infection, the interaction map identifies all connections potentially needed for the virus to replicate and escape host defence.

### Example 2

Hepatitis C virus (HCV) infected patients with high serum levels of bile acids (BAs) usually fail to respond to antiviral therapy. The role of BAs on HCV RNA replication was thus assessed. BAs, especially chenodeoxycholate and deoxycholate, up-regulated HCV RNA replication by more than tenfold. Only free but not conjugated BAs were active, suggesting that their effect was mediated by a nuclear receptor. Only farnesoid X receptor (FXR) ligands stimulated HCV replication while FXR silencing and FXR antagonism by guggulsterone blocked the up-regulation induced by BAs. Furthermore, guggulsterone alone inhibited basal level of HCV replication by tenfold. Modulation of HCV replication by FXR ligands occurred in the same proportion in presence or absence of type I interferon, suggesting a mechanism of action independent of this control of viral replication. Thus, exposure to BAs increases HCV replication by a novel mechanism involving activation of the nuclear receptor FXR.

This raises the possibility that the virus could directly interfere with FXR to favour its replication. To complete the proteome-wide screening of HCV proteins cellular interactors, we performed a specific screening focused on FXR to identify all potential interactions between this receptor and any viral proteins.

### Material

### Bacteria

Escherichia coli competent bacteria (OneShot® Top10, Invitrogen) (F-mcrA Δ (mrr-hsdRMS-mcrBC) φ80lacZΔM15 ΔlacX74 recA1 araD139 Δ (ara-leu), 7697 galU galK rpsL (StrR) endA1 nupG).

### Yeasts

We used the following strains: AH109 et Y187 (Clontech) Saccharomyces cerevisiae with the following genotype:

AH109: MATa, trp1-901, leu2-3, 112, ura3-52, his3-200, gal4Δ, gal80Δ, LYS2::GAL1_{UAS}-GAL1_{TATA}-HIS3, GAL2_{UAS}-GAL2_{TATA}-ADE2, ura3::MEL1_{UAS}-MEL1_{TATA}-lacZ.
Y187: MATa, ura3-52, his3-200, ade2-101, trp1-901, leu2-3, 112, met-, gal4Δ, gal80A, MEL1, URA3::GAL1UAS-GAL1TATA-lacZ.

### Human Cells

Hek-293T: human embryonic kidney cells expressing large T antigen.
Identification of HCV Proteins Interacting with FXR Using Yeast Two Hybrid Matrix

Our previous work indicated that HCV replication can be under the control of FXR activity. In order to identify viral components that could interfere with FXR activity, we search for interaction between viral proteins and FXR. This was done by pairwise interaction screening with the yeast-two-hybrid method. All 10 viral proteins have been tested and data are summarized in the following table:

| E1 | E2 | Core | P7 | NS2 | NS3 | NS4A | NS4B | NS5A | NS5B |
|---|---|---|---|---|---|---|---|---|---|
| − | − | − | − | − | + | − | − | + | − |

### Pulldown Experiments

The data have been confirmed by GST-pull down in mammal cells. Viral proteins in fusion with GST were co-transfected with FXR tagged with 3×Flag in Hek-293T cells. 48 h latter, cells were lysed, precipitation was performed with glutathion sepharose and subjected to electrophoresis and western blot with anti-Flag (to reveal FXR) or anti-GST antibodies coupled to peroxidase. Co-precipitations of FXR with NS3 and NS5A were positive confirming direct interaction of these viral proteins with FXR. Co-precipitations of FXR with all other viral proteins were negative confirming that these proteins do not interact with FXR.

### REFERENCES

Throughout this application, various references describe the state of the art to which this invention pertains. The disclosures of these references are hereby incorporated by reference into the present disclosure.
1. F. Negro, J. Hepatol. 45, 514 (2006).
2. D. Moradpour, F. Penin, C. M. Rice, Nat. Rev. Microbiol. 5, 453 (2007).
3. N. Appel, T. Schaller, F. Penin, R. Bartenschlager, J. Biol. Chem. 281, 9833 (2006).
4. D. B. Strader, T. Wright, D. L. Thomas, L. B. Seeff, Hepatology 39, 1147 (2004).
5. A. Lonardo et al., Gastroenterology 126, 586 (2004).
6. S. L. Tan, G. Ganji, B. Paeper, S. Proll, M. G. Katze, Nat. Biotechnol. 25, 1383 (2007).
7. M. A. Calderwood et al., Proc. Natl. Acad. Sci. U.S.A. 104, 7606 (2007).
8. P. Uetz et al., Science 311, 239 (2006).
9. M. D. Dyer, T. M. Murali, B. W. Sobral, PLoS Pathog. 4, e32 (2008).
10. A. J. Walhout et al., Methods Enzymol. 328, 575 (2000).
11. G. D. Bader, D. Betel, C. W. Hogue, Nucleic Acids Res. 31, 248 (2003).
12. J. F. Rual et al., Nature 437, 1173 (2005).
13. H. Huang, B. M. Jedynak, J. S. Bader, PLoS Comput. Biol. 3, e214 (2007).
14. T. K. Gandhi et al., Nat. Genet. 38, 285 (March, 2006).
15. U. Stelzl et al., Cell 122, 957 (2005).
16. P. Y. Lozach et al., J. Biol. Chem. 278, 20358 (2003).
17. N. L. Kedersha, L. H. Rome, J. Cell Biol. 103, 699 (1986).
18. F. Ramirez, A. Schlicker, Y. Assenov, T. Lengauer, M. Albrecht, Proteomics 7, 2541 (2007).
19. P. Hernandez et al., BMC Genomics 8, 185 (2007).
20. D. Ekman, S. Light, A. K. Bjorklund, A. Elofsson, Genome Biol. 7, R45 (2006).
21. X. He, J. Zhang, PLoS Genet. 2, e88 (2006).
22. K. I. Goh et al., Proc. Natl. Acad. Sci. U.S.A. 104, 8685 (2007).
23. S. Wachi, K. Yoneda, R. Wu, Bioinformatics 21, 4205 (2005).
24. M. P. Joy, A. Brock, D. E. Ingber, S. Huang, J. Biomed. Biotechnol. 2005, 96 (2005).
25. R. D'Souza, C. A. Sabin, G. R. Foster, Am. J. Gastroenterol. 100, 1509 (2005).
26. M. Romero-Gomez et al., Gastroenterology 128, 636 (2005).
27. T. Poynard et al., Gastroenterology 122, 1303 (2002).
28. D. Schuppan, A. Krebs, M. Bauer, E. G. Hahn, Cell Death Differ. 10 Suppl 1, S59 (2003).
29. M. Romero-Gomez, World J. Gastroenterol. 12, 7075 (2006).
30. S. K. Sahu, S. N. Gummadi, N. Manoj, G. K. Aradhyam, Arch. Biochem. Biophys. 462, 103 (2007).
31. B. Dong et al., J. Virol. 78, 8983 (2004).
32. T. Wiedmer et al., Proc. Natl. Acad. Sci. U.S.A. A 101, 13296 (2004).
33. C. Q. He, N. Z. Ding, W. Fan, Mol. Cell. Biochem. 308, 247 (2008).
34. K. Kurisaki et al., Mol. Cell. Biol. 23, 4494 (2003).
35. R. T. Mai et al., Oncogene 25, 448 (2006).
36. V. Pazienza et al., Hepatology 45, 1164 (2007).
37. Y. Shintani et al., Gastroenterology 126, 840 (2004).
38. T. Kawaguchi et al., Am. J. Pathol. 165, 1499 (2004).
39. M. J. Lehmann, N. M. Sherer, C. B. Marks, M. Pypaert, W. Mothes, J. Cell. Biol. 170, 317 (2005).
Chien C T, Bartel P L, Sternglanz R, Fields S. The two-hybrid system: a method to identify and clone genes for proteins that interact with a protein of interest. Proc Natl Acad Sci USA. 1991 Nov. 1; 88(21):9578-82.
Edwards P R, Leatherbarrow R J. Determination of association rate constants by an optical biosensor using initial rate analysis. Anal Biochem. 1997 Mar. 1; 246(1):1-6.
Fearon E R, Finkel T, Gillison M L, Kennedy S P, Casella J F, Tomaselli G F, Morrow J S, Van Dang C. Karyoplasmic interaction selection strategy: a general strategy to detect protein-protein interactions in mammalian cells. Proc Natl Acad Sci USA. 1992 Sep. 1; 89(17):7958-62.
Fields S, Song O. A novel genetic system to detect protein-protein interactions. Nature. 1989 Jul. 20; 340(6230):245-6.
Kurien B T, Scofield R H. Western blotting. Methods. 2006 April; 38(4):283-93.
Leblanc V, Delaunay V, Claude Lelong J, Gas F, Mathis G, Grassi J, May E. Homogeneous time-resolved fluorescence assay for identifying p53 interactions with its protein partners, directly in a cellular extract. Anal Biochem. 2002 Sep. 15; 308(2):247-54.
Rybicki E P, von Wechmar M B. Enzyme-assisted immune detection of plant virus proteins electroblotted onto nitrocellulose paper. J Virol Methods. 1982 December; 5(5-6):267-78
Szabo A, Stolz L, Granzow R. Surface plasmon resonance and its use in biomolecular interaction analysis (BIA). Curr Opin Struct Biol. 1995 October; 5(5):699-705.
Towbin H, Staehelin T, Gordon J. Electrophoretic transfer of proteins from polyacrylamide gels to nitrocellulose sheets: procedure and some applications. Proc Natl Acad Sci USA. 1979 September; 76(9):4350-4.
Vasavada H A, Ganguly S, Germino F J, Wang Z X, Weissman S M. A contingent replication assay for the detection of protein-protein interactions in animal cells. Proc Natl Acad Sci USA. 1991 Dec. 1; 88(23)10686-90.
Chien C T, Bartel P L, Sternglanz R, Fields S. The two-hybrid system: a method to identify and clone genes for proteins that interact with a protein of interest. Proc Natl Acad Sci USA. 1991 Nov. 1; 88(21):9578-82.
Edwards P R, Leatherbarrow R J. Determination of association rate constants by an optical biosensor using initial rate analysis. Anal Biochem. 1997 Mar. 1; 246(1):1-6.
Fearon E R, Finkel T, Gillison M L, Kennedy S P, Casella J F, Tomaselli G F, Morrow J S, Van Dang C. Karyoplasmic interaction selection strategy: a general strategy to detect protein-protein interactions in mammalian cells. Proc Natl Acad Sci USA. 1992 Sep. 1; 89(17):7958-62.
Fields S, Song O. A novel genetic system to detect protein-protein interactions. Nature. 1989 Jul. 20; 340(6230):245-6.
Kurien B T, Scofield R H. Western blotting. Methods. 2006 April; 38(4):283-93.
Leblanc V, Delaunay V, Claude Lelong J, Gas F, Mathis G, Grassi J, May E. Homogeneous time-resolved fluorescence assay for identifying p53 interactions with its protein partners, directly in a cellular extract. Anal Biochem. 2002 Sep. 15; 308(2):247-54.
Rybicki E P, von Wechmar M B. Enzyme-assisted immune detection of plant virus proteins electroblotted onto nitrocellulose paper. J Virol Methods. 1982 December; 5(5-6):267-78
Szabo A, Stolz L, Granzow R. Surface plasmon resonance and its use in biomolecular interaction analysis (BIA). Curr Opin Struct Biol. 1995 October; 5(5):699-705.
Towbin H, Staehelin T, Gordon J. Electrophoretic transfer of proteins from polyacrylamide gels to nitrocellulose sheets: procedure and some applications. Proc Natl Acad Sci USA. 1979 September; 76(9):4350-4.
Vasavada H A, Ganguly S, Germino F J, Wang Z X, Weissman S M. A contingent replication assay for the detection of protein-protein interactions in animal cells. Proc Natl Acad Sci USA. 1991 Dec. 1; 88(23)10686-90.

### Tables

TABLE S1

| | Human protein | | | | IMAP LCI | | | |
|---|---|---|---|---|---|---|---|---|
| | (NCBI) | | | | Text | | | |
| HCV | | Gene | IMAP Y2H | | mining | BIND | | Y2H |
| protein | Gene name | ID | IMAP1 | IMAP2 | (PMID) | (PMID) | CoAP | matrices |
| CORE | ACP1 | 52 | | | 15846844 | | | |
| CORE | AGRN | 375790 | 1 | | | | | + |
| CORE | APOA2 | 336 | | | 10498661\| | | | |
| | | | | | 15732001 | | | |
| CORE | BCAR1 | 9564 | 1 | | | | | + |
| CORE | C1QBP | 708 | | | 11086025\| | | | |
| | | | | | 11792059\| | | | |
| | | | | | 14517080\| | | | |
| | | | | | 15292184\| | | | |
| | | | | | 16306613 | | | |
| CORE | CCNH | 902 | | | 14711830 | | | |
| CORE | CD68 | 968 | 1 | | | | | + |
| CORE | CDKN1A | 1026 | | | | 10873631 | | |
| CORE | CFL1 | 1072 | | | 15846844 | | | |
| CORE | COL4A2 | 1284 | 1 | | | | | + |
| CORE | CREBBP | 1387 | | | | 15380363 | | |
| CORE | DDX3X | 1654 | | 1 | 10329544 | 10329544\| | NA | |
| | | | | | | 10336476\| | | |
| | | | | | | 1848704 | | |
| CORE | DDX3Y | 8653 | | 3 | | | | |
| CORE | DDX5 | 1655 | | | 15846844 | | | |
| CORE | DICER1 | 23405 | | | 16530526 | | | |
| CORE | EGFL7 | 51162 | | 1 | | | NA | |
| CORE | EP300 | 2033 | | | 15380363 | 15380363 | | |
| CORE | FADD | 8772 | | | 11336543 | | | |
| CORE | FAS | 355 | | | 12919737 | | | |
| CORE | FBLN2 | 2199 | 1 | | | | | + |
| CORE | FBLN5 | 10516 | 1 | | | | | + |
| CORE | FKBP7 | 51661 | | | 15846844 | | | |
| CORE | FUNDC2 | 65991 | | | 12665903 | | | |
| CORE | GAPDH | 2597 | 1 | | | | NA | + |
| CORE | GRN | 2896 | 1 | | | | | + |
| CORE | HBXAP | 51773 | | | 12401801 | | | |
| CORE | HIVEP2 | 3097 | | 2 | | | | |
| CORE | HLA-A | 3105 | | | 15681828 | | | |
| CORE | HLA-E | 3133 | | | 15681828 | | | |
| CORE | HNRPK | 3190 | | 14 | 9651361 | 9651361 | | |
| CORE | HOXD8 | 3234 | 1 | | | | | + |
| CORE | HSPD1 | 3329 | | | 15846844 | | | |
| CORE | JAK1 | 3716 | | | 12764155 | | | |
| CORE | JAK2 | 3717 | | | 12764155 | | | |
| CORE | KRT18 | 3875 | | | 15846844 | | | |
| CORE | KRT19 | 3880 | | | 15846844 | | | |
| CORE | KRT8 | 3856 | | | 15846844 | | | |
| CORE | LPXN | 9404 | 1 | | | | | + |
| CORE | LRRTM1 | 347730 | 1 | | | | | + |
| CORE | LTBP4 | 8425 | 4 | | | | | + |
| CORE | LTBR | 4055 | | | 8995654\| | 9371602\| | | |
| | | | | | 9371602 | 2117749 | | |
| CORE | MAGED1 | 9500 | 1 | 2 | | | NA | + |
| CORE | MEGF6 | 1953 | 1 | | | | | + |
| CORE | MMRN2 | 79812 | 1 | | | | | + |
| CORE | NPM1 | 4869 | | | 16170350 | | | |
| CORE | NR4A1 | 3164 | 31 | | | | | + |
| CORE | PABPN1 | 8106 | | 1 | | | NA | |
| CORE | PAK4 | 10298 | 1 | | | | | + |
| CORE | PLSCR1 | 5359 | 1 | | | | | + |
| CORE | PML | 5371 | | | 16322229 | | | |
| CORE | PSME3 | 10197 | | | 12970408 | | | |
| CORE | RNF31 | 55072 | 1 | | | | | + |
| CORE | RXRA | 6256 | | | 11915042 | | | |
| CORE | SETD2 | 29072 | 5 | | | | | + |
| CORE | SLC22A7 | 10864 | | | 15846844 | | | |
| CORE | SLC31A2 | 1318 | 1 | | | | | + |
| CORE | SMAD3 | 4088 | | | 15334054\| | 15334054 | | |
| | | | | | 16007207 | | | |
| CORE | STAT1 | 6772 | | | 15825084 | | | |
| CORE | STAT3 | 6774 | | | 12208879 | | | |
| CORE | TAF11 | 6882 | | | 10924497 | | | |
| CORE | TATDN1 | 83940 | | | 15846844 | | | |
| CORE | TBP | 6908 | | | 14730212 | | | |
| CORE | TGFBR1 | 7046 | | | 16407286 | | | |
| CORE | TNFRSF1A | 7132 | | | 9557650\| | 11226577 | | |
| | | | | | 11226577\| | | | |
| | | | | | 11336543 | | | |
| CORE | TP53 | 7157 | | | 10544138\| | 10544138\| | | |
| | | | | | 10924497 | 12730672 | | |
| CORE | TP53BP2 | 7159 | | | 14985081 | 14985081 | | |
| CORE | TP73 | 7161 | | | 12730672 | 12730672 | | |
| CORE | TSN | 7247 | | | 12133464\| | 12532453 | | |
| | | | | | 12532453 | | | |
| CORE | TXNL2 | 10539 | | | 15846844 | | | |
| CORE | VIM | 7431 | | | 15846844 | | | |
| CORE | VWF | 7450 | 2 | | | | | + |
| CORE | YWHAB | 7529 | | | | 10644344 | | |
| CORE | YWHAE | 7531 | | | 10644344 | 10644344 | | |
| CORE | YWHAZ | 7534 | | | | 10644344 | | |
| CORE | YY1 | 7528 | | | 16170350 | | | |
| CORE | ZNF271 | 10778 | 1 | | | | | + |
| E1 | CALR | 811 | | | 9557669\| | | | |
| | | | | | 11602760 | | | |
| E1 | CANX | 821 | | | 9557669\| | | | |
| | | | | | 11602760 | | | |
| E1 | CD209 | 30835 | | | 12634366\| | 12634366\| | | |
| | | | | | 15254204 | 15254204 | | |
| E1 | CLEC4M | 10332 | | | 12634366 | 12634366 | | |
| E1 | HSPA5 | 3309 | | | 9557669 | | | |
| E1 | JUN | 3725 | 1 | | | | | + |
| E1 | LTF | 4057 | | | 9223490 | 9223490\| | | |
| | | | | | | 9143310 | | |
| E1 | NR4A1 | 3164 | 14 | | | | | + |
| E1 | PFN1 | 5216 | 1 | | | | | + |
| E1 | SETD2 | 29072 | 2 | | | | | + |
| E1 | TMSB4X | 7114 | 1 | | | | | + |
| E2 | CALR | 811 | | | 9557669 | | | |
| E2 | CANX | 821 | | | 9557669 | | | |
| E2 | CD209 | 30835 | | | 12609975\| | 12634366\| | | |
| | | | | | 12634366\| | 15254204 | | |
| | | | | | 15166245\| | | | |
| | | | | | 15254204 | | | |
| E2 | CD81 | 975 | | | 9794763\| | 12604806\| | | |
| | | | | | 12604806\| | 10846074\| | | |
| | | | | | 16600629 | 11080483\| | | |
| | | | | | | 12522210\| | | |
| | | | | | | 10729140 | | |
| E2 | CLEC4M | 10332 | | | 12609975\| | 12634366 | | |
| | | | | | 12634366\| | | | |
| | | | | | 15166245 | | | |
| E2 | EIF2AK2 | 5610 | | | 10390359\| | | | |
| | | | | | 11494536\| | | | |
| | | | | | 11773402 | | | |
| E2 | EIF2AK3 | 9451 | | | 12610133 | | | |
| E2 | HOXD8 | 3234 | 1 | | | | | + |
| E2 | HSPA5 | 3309 | | | 9557669 | | | |
| E2 | ITGB1 | 3688 | 1 | | | | | + |
| E2 | KIAA1411 | 57579 | 1 | | | | | + |
| E2 | LOC730765 | 730765 | 1 | | | | | + |
| E2 | LTF | 4057 | | | 9223490 | 12522210\| | | |
| | | | | | | 9223490 | | |
| E2 | NR4A1 | 3164 | 6 | | | | | + |
| E2 | PSMA6 | 5687 | 1 | | | | | + |
| E2 | SCARB1 | 949 | | | 12356718\| | | | |
| | | | | | 15325070\| | | | |
| | | | | | 15632171\| | | | |
| | | | | | 16099909 | | | |
| E2 | SDC2 | 6383 | | | 12867431 | | | |
| E2 | SETD2 | 29072 | 1 | | | | | + |
| E2 | SMEK2 | 57223 | 1 | | | | | + |
| E2 | TF | 7018 | | | | 12522210 | | |
| F | AGT | 183 | | | 16237761 | | | |
| F | AZGP1 | 563 | | | 16237761 | | | |
| F | CTSB | 1508 | | | 16237761 | | | |
| F | MPDU1 | 9526 | | | 16237761 | | | |
| F | RAB14 | 51552 | | | 16237761 | | | |
| F | SERPINC1 | 462 | | | 16237761 | | | |
| F | ST3GAL1 | 6482 | | | 16237761 | | | |
| F | VTN | 7431 | | | 16237761 | | | |
| F | ZG16 | 123887 | | | 16237761 | | | |
| NS3 | C14orf173 | 64423 | | 6 | | | NA | |
| NS2 | C7 | 730 | 1 | | | | | + |
| NS3 | CCDC21 | 64793 | | 2 | | | + | |
| NS2 | CIDEB | 27141 | | | 12595532 | 12595532 | | |
| NS2 | FBLN5 | 10516 | 1 | | | | | + |
| NS2 | HOXD8 | 3234 | 1 | | | | | + |
| NS2 | NR4A1 | 3164 | 5 | | | | | + |
| NS2 | POU3F2 | 5454 | 1 | | | | | + |
| NS3 | PRRC1 | 133619 | | 1 | | | | |
| NS2 | SETD2 | 29072 | 1 | | | | | + |
| NS2 | TRIM27 | 5987 | | 1 | | | | |
| NS3 | ACTN1 | 87 | 9 | | | | | + |
| NS3 | ACTN2 | 88 | 10 | 6 | | | − | + |
| NS3 | AEBP1 | 165 | 2 | | | | | + |
| NS3 | ANKRD12 | 23253 | 1 | | | | | + |
| NS3 | ANKRD28 | 23243 | 1 | | | | | + |
| NS3 | ARFIP2 | 23647 | 1 | | | | | + |
| NS3 | ARHGEF6 | 9459 | 1 | | | | | + |
| NS3 | ARNT | 405 | 1 | | | | | + |
| NS3 | ARS2 | 51593 | 4 | | | | | + |
| NS3 | ASXL1 | 171023 | 1 | | | | | + |
| NS3 | B2M | 567 | | 1 | | | | |
| NS3 | BCAN | 63827 | 2 | | | | | + |
| NS3 | BCKDK | 10295 | | 1 | | | + | |
| NS3 | BCL2A1 | 597 | | 1 | | | | |
| NS3 | BCL6 | 604 | 1 | | | | | + |
| NS3 | BZRAP1 | 9256 | 1 | | | | | + |
| NS3 | C10orf18 | 54906 | 3 | | | | | + |
| NS3 | C10orf6 | 55719 | 1 | | | | | + |
| NS3 | C12orf41 | 54934 | 1 | | | | | + |
| NS3 | C16orf7 | 9605 | | 1 | | | | |
| NS3 | C1orf165 | 79656 | 1 | | | | | + |
| NS3 | C1orf94 | 84970 | 1 | | | | | + |
| NS3 | C9orf30 | 91283 | 1 | | | | | + |
| NS3 | CALCOCO2 | 10241 | 5 | 2 | | | + | + |
| NS3 | CBY1 | 25776 | 2 | | | | | + |
| NS3 | CCDC37 | 348807 | 1 | | | | | + |
| NS3 | CCDC52 | 152185 | 1 | | | | | + |
| NS3 | CCDC66 | 285331 | 1 | 4 | | | | + |
| NS3 | CCDC95 | 283899 | 1 | | | | | + |
| NS3 | CCHCR1 | 54535 | 1 | 2 | | | | + |
| NS3 | CD5L | 922 | 1 | 1 | | | | + |
| NS3 | CDC23 | 8697 | 1 | | | | | + |
| NS3 | CELSR2 | 1952 | 1 | | | | | + |
| NS3 | CEP152 | 22995 | 1 | | | | | + |
| NS3 | CEP192 | 55125 | 1 | | | | | + |
| NS3 | CFP | 5199 | 4 | | | | | + |
| NS3 | CHPF | 79586 | 1 | | | | | + |
| NS3 | CORO1B | 57175 | 8 | | | | | + |
| NS3 | COX3 | 4514 | | 1 | | | NA | |
| NS3 | CSNK2B | 1460 | 3 | | | | | + |
| NS3 | CTGF | 1490 | 1 | | | | | + |
| NS3 | CXorf45 | 79868 | 1 | | | | | + |
| NS3 | DEAF1 | 10522 | 6 | | | | | + |
| NS3 | DES | 1674 | 1 | | | | | + |
| NS3 | DLAT | 1737 | | 8 | | | NA | |
| NS3 | DOCK7 | 85440 | | 2 | | | | |
| NS3 | DPF1 | 8193 | 1 | | | | | + |
| NS3 | DPP7 | 29952 | 1 | | | | | + |
| NS3 | EEF1A1 | 1915 | | 1 | | | NA | |
| NS3 | EFEMP1 | 2202 | 10 | | | | | + |
| NS3 | EFEMP2 | 30008 | 3 | | | | | + |
| NS3 | EIF1 | 10209 | | 1 | | | | |
| NS3 | EIF4ENIF1 | 56478 | 2 | 3 | | | + | + |
| NS3 | ERC1 | 23085 | | | 16033967 | | | |
| NS3 | FAM120B | 84498 | 1 | | | | | + |
| NS3 | FAM65A | 79567 | | 1 | | | | |
| NS3 | FBF1 | 85302 | | 3 | | | | |
| NS3 | FBLN1 | 2192 | 8 | | | | | + |
| NS3 | FBLN2 | 2199 | 9 | | | | | + |
| NS3 | FBLN5 | 10516 | 32 | | | | | + |
| NS3 | FBN1 | 2200 | 1 | | | | | + |
| NS3 | FBN3 | 84467 | 2 | | | | | + |
| NS3 | FES | 2242 | 1 | | | | | + |
| NS3 | FIGNL1 | 63979 | 1 | | | | NA | + |
| NS3 | FLAD1 | 80308 | 1 | | | | | + |
| NS3 | FLJ11286 | 55337 | 1 | 3 | | | | + |
| NS3 | FN1 | 2335 | 4 | | | | | + |
| NS3 | FRMPD4 | 9758 | 1 | | | | | + |
| NS3 | FRS3 | 10817 | 2 | 1 | | | + | + |
| NS3 | FTH1 | 2495 | 2 | | | | | + |
| NS3 | FUCA2 | 2519 | 2 | | | | | + |
| NS3 | GAA | 2548 | 2 | | | | | + |
| NS3 | GBP2 | 2634 | | 26 | | | + | |
| NS3 | GFAP | 2670 | 43 | | | | NA | + |
| NS3 | GNB2 | 2783 | 1 | | | | | + |
| NS3 | GON4L | 54856 | 1 | | | | | + |
| NS3 | HIST3H2BB | 128312 | | | | 10405893\| | | |
| | | | | | | 8647104 | | |
| NS3 | HIST4H4 | 121504 | | | | 8647104 | | |
| NS3 | HIVEP2 | 3097 | 3 | 5 | | | | + |
| NS3 | HNRPK | 3190 | | 1 | | | | |
| NS3 | NAPL1L2 | 4674 | | 1 | | | | |
| NS3 | HOMER3 | 9454 | 4 | 1 | | | NA | + |
| NS3 | HRMT1L2 | 3276 | | | 11483748 | | | |
| NS3 | IKBKE | 9641 | | | 15841462 | | | |
| NS3 | IQWD1 | 55827 | 2 | | | | | + |
| NS3 | ITGB4 | 3691 | 7 | | | | | + |
| NS3 | JAG2 | 3714 | 1 | | | | | + |
| NS3 | JUN | 3725 | 8 | | | | | + |
| NS3 | KHDRBS1 | 10657 | 4 | | | | | + |
| NS3 | KIAA1549 | 57670 | 1 | | | | | + |
| NS3 | KIF17 | 57576 | 2 | | | | NA | + |
| NS3 | KIF7 | 374654 | 1 | | | | | + |
| NS3 | KPNA1 | 3836 | | 8 | | | + | |
| NS3 | L3MBTL3 | 84456 | 4 | 8 | | | NA | + |
| NS3 | LAMA5 | 3911 | 1 | | | | | + |
| NS3 | LAMB2 | 3913 | 1 | | | | | + |
| NS3 | LAMC3 | 10319 | 1 | | | | | + |
| NS3 | LDB1 | 8861 | 1 | | | | | + |
| NS3 | LOC728302 | 728302 | 2 | 3 | | | | + |
| NS3 | LRRC7 | 57554 | | 6 | | | | |
| NS3 | LRRCC1 | 85444 | 1 | | | | | + |
| NS3 | LTBP4 | 8425 | 2 | | | | | + |
| NS3 | LZTS2 | 84445 | 8 | | | | | + |
| NS3 | MAGED1 | 9500 | 1 | | | | NA | + |
| NS3 | MAPK7 | 5598 | 4 | | | | | + |
| NS3 | MBP | 4155 | | | | 8647104 | | |
| NS3 | MEGF8 | 1954 | 2 | | | | | + |
| NS3 | MLLT4 | 4301 | 2 | | | | | + |
| NS3 | MLXIP | 22877 | 1 | | | | | + |
| NS3 | MORC4 | 79710 | 1 | | | | | + |
| NS3 | MORF4L1 | 10933 | | 1 | | | | |
| NS3 | MVP | 9961 | 1 | | | | | + |
| NS3 | NAP1L1 | 4673 | | 1 | | | NA | |
| NS3 | NCAN | 1463 | 1 | | | | | + |
| NS3 | NDC80 | 10403 | | 1 | | | | |
| NS3 | NEFL | 4747 | 1 | | | | | + |
| NS3 | NEFM | 4741 | 1 | | | | | + |
| NS3 | NELL1 | 4745 | 3 | | | | | + |
| NS3 | NELL2 | 4753 | 11 | | | | | + |
| NS3 | NID1 | 4811 | 2 | | | | | + |
| NS3 | NID2 | 22795 | 2 | | | | | + |
| NS3 | NOTCH1 | 4851 | 2 | | | | | + |
| NS3 | N-PAC | 84656 | 2 | 11 | | | | + |
| NS3 | NUP62 | 23636 | 3 | | | | | + |
| NS3 | OBSCN | 84033 | 1 | | | | | + |
| NS3 | PARP4 | 143 | 8 | | | | | + |
| NS3 | PCYT2 | 5833 | 1 | | | | | + |
| NS3 | PDE4DIP | 9659 | 3 | | | | | + |
| NS3 | PDLIM5 | 10611 | 1 | | | | | + |
| NS3 | PICK1 | 9463 | 3 | | | | | + |
| NS3 | PKNOX1 | 5316 | 1 | | | | | + |
| NS3 | PLEKHG4 | 25894 | 1 | | | | | + |
| NS3 | PNPLA8 | 50640 | 1 | | | | | + |
| NS3 | PRKACA | 5566 | | | | 9060639\| | | |
| | | | | | | 8647104 | | |
| NS3 | PRM1 | 5619 | | | | 8647104 | | |
| NS3 | PRMT1 | 3276 | | | | 9371600\| | | |
| | | | | | | 11483748\| | | |
| | | | | | | 9188558 | | |
| NS3 | PSMB8 | 5696 | | | 15303969 | 15303969\| | | |
| | | | | | | 11556407 | | |
| NS3 | PSMB9 | 5698 | | 1 | | | + | |
| NS3 | PSME3 | 10197 | 10 | | | | | + |
| NS3 | PTBP2 | 58155 | | | 15823607 | | | |
| NS3 | PTPRN2 | 5799 | 1 | | | | | + |
| NS3 | RABEP1 | 9135 | 2 | 1 | | | NA | + |
| NS3 | RAI14 | 26064 | 1 | 2 | | | − | + |
| NS3 | RASAL2 | 9462 | | 3 | | | + | |
| NS3 | RBM4 | 5936 | 1 | | | | | + |
| NS3 | RCN3 | 57333 | 2 | | | | | + |
| NS3 | RGNEF | 64283 | 1 | | | | | + |
| NS3 | RICS | 9743 | 3 | 1 | | | | + |
| NS3 | RINT1 | 60561 | 1 | | | | | + |
| NS3 | RNF31 | 55072 | 1 | | | | | + |
| NS3 | ROGDI | 79641 | | 1 | | | | |
| NS3 | RP11- | 340533 | | 1 | | | | |
| | 130N24.1 | | | | | | | |
| NS3 | RSHL2 | 83861 | 1 | | | | | + |
| NS3 | RUSC2 | 9853 | 21 | 1 | | | | + |
| NS3 | SBF1 | 6305 | 1 | | | | | + |
| NS3 | SDCCAG8 | 10806 | 1 | | | | | + |
| NS3 | SECISBP2 | 79048 | 1 | | | | | + |
| NS3 | SEPT10 | 151011 | 1 | | | | | + |
| NS3 | SERPINF2 | 5345 | | | 10570951 | 10570951 | | |
| NS3 | SERPING1 | 710 | | | 10570951 | 10570951 | | |
| NS3 | SERTAD1 | 29950 | 3 | | | | | + |
| NS3 | SESTD1 | 91404 | 1 | | | | | + |
| NS3 | SF3B2 | 10992 | 1 | | | | | + |
| NS3 | SIAH1 | 6477 | 1 | | | | | + |
| NS3 | PRMT5 | 10419 | | | | 11152681 | | |
| NS3 | SLIT1 | 6585 | 7 | | | | | + |
| NS3 | SLIT2 | 9353 | 5 | | | | | + |
| NS3 | SLIT3 | 6586 | 4 | | | | | + |
| NS3 | SMAD3 | 4088 | | | 15334054 | 15334054 | | |
| NS3 | SMURF2 | 64750 | | 4 | | | + | |
| NS3 | SNRPD1 | 6632 | | | 14524621 | 14524621 | | |
| NS3 | SNX4 | 8723 | 1 | | | | | + |
| NS3 | SPOCK3 | 50859 | 1 | | | | | + |
| NS3 | SPON1 | 10418 | 1 | | | | | + |
| NS3 | SRPX2 | 27286 | 1 | | | | | + |
| NS3 | SSX2IP | 117178 | 3 | | | | | + |
| NS3 | STAB1 | 23166 | 4 | | | | | + |
| NS3 | STAT3 | 6774 | 1 | | | | | + |
| NS3 | SVEP1 | 79987 | 3 | | | | | + |
| NS3 | SYNE1 | 23345 | | 4 | | | | |
| NS3 | SYNPO2 | 171024 | | 1 | | | | |
| NS3 | TAF1 | 6872 | 2 | | | | | + |
| NS3 | TBC1D2B | 23102 | 1 | | | | | + |
| NS3 | TBK1 | 29110 | | | 15841462 | | | |
| NS3 | TBXAS1 | 6916 | | 1 | | | | |
| NS3 | TGFB1I1 | 7041 | 1 | | | | | + |
| NS3 | THAP1 | 55145 | 1 | | | | | + |
| NS3 | TICAM1 | 148022 | | | 15767257 | | | |
| NS3 | TMEM63B | 55362 | | 1 | | | | |
| NS3 | TP53 | 7157 | | | | 9827557 | | |
| NS3 | TRIM23 | 373 | 5 | | | | | + |
| NS3 | TRIM27 | 5987 | 1 | | | | | + |
| NS3 | TRIO | 7204 | 1 | 1 | | | | + |
| NS3 | TRIP11 | 9321 | 1 | | | | | + |
| NS3 | TXNDC11 | 51061 | 2 | | | | NA | + |
| NS3 | UBE1C | 9039 | | 1 | | | NA | |
| NS3 | USHBP1 | 83878 | 1 | | | | | + |
| NS3 | UXT | 8409 | 1 | | | | | + |
| NS3 | VCAN | 1462 | 1 | | | | | + |
| NS3 | VIM | 7431 | 32 | | | | | + |
| NS3 | VWF | 7450 | 26 | | | | | + |
| NS3 | XAB2 | 56949 | 2 | | | | | + |
| NS3 | XRN2 | 22803 | 1 | | | | | + |
| NS3 | YY1AP1 | 55249 | 7 | | | | | + |
| NS3 | ZBTB1 | 22890 | 1 | | | | | + |
| NS3 | ZCCHC7 | 84186 | 2 | 1 | | | | + |
| NS3 | ZHX3 | 23051 | 4 | | | | | + |
| NS3 | ZMYM2 | 7750 | | 2 | | | | |
| NS3 | ZNF281 | 23528 | 1 | | | | | + |
| NS3 | ZNF410 | 57862 | 1 | | | | | + |
| NS3 | ZZZ3 | 26009 | 1 | | | | | + |
| NS4A | CREB3 | 10488 | | 1 | | | NA | |
| NS4A | ELAC2 | 60528 | 1 | | | | | + |
| NS4A | HOXD8 | 3234 | 1 | | | | | + |
| NS4A | NR4A1 | 3164 | 3 | | | | | + |
| NS4A | TRAF3IP3 | 80342 | | 1 | | | NA | |
| NS4A | UBQLN1 | 29979 | 2 | | | | | + |
| NS4B | CREBL1 | 1388 | | | | 12445808 | | |
| NS5A | ACLY | 47 | | 1 | | | | |
| NS5A | AHNAK | 79026 | | | 15607035 | 15607035 | | |
| NS5A | AHSA1 | 3320 | | | | 17616579 | | |
| NS5A | AKT1 | 207 | | | | 17616579 | | |
| NS5A | APOA1 | 335 | | | | 11878923 | | |
| NS5A | APOE | 348 | | | | 15326295 | | |
| NS5A | ARFIP1 | 27236 | | 9 | | | | |
| NS5A | AXIN1 | 8312 | | 1 | | | | |
| NS5A | BAX | 581 | | | 12925958 | | | |
| NS5A | BIN1 | 274 | 16 | 14 | 12604805\| | 12604805\| | + | + |
| | | | | | 16139795 | 10390360 | | |
| NS5A | C10orf30 | 222389 | | 1 | | | | |
| NS5A | C9ORF6 | 54942 | | | 15607035 | 15607035 | | |
| NS5A | CADPS | 8618 | | 1 | | | | |
| NS5A | CADPS2 | 93664 | | 1 | | | | |
| NS5A | CCDC100 | 153241 | | 1 | | | | |
| NS5A | CCDC86 | 79080 | | | 15607035 | | | |
| NS5A | CDK1 | 983 | | | 11278402 | | | |
| NS5A | CDK6 | 1021 | | | | 17616579 | | |
| NS5A | CENPC1 | 1060 | | 1 | | | NA | |
| NS5A | CENTD2 | 116985 | | | 15607035 | 15607035 | | |
| NS5A | CEP250 | 11190 | | 15 | | | | |
| NS5A | CEP57 | 9702 | | | 15607035 | 15607035 | | |
| NS5A | CEP63 | 80254 | | 1 | | | | |
| NS5A | CRABP1 | 1381 | | | 15607035 | 15607035 | | |
| NS5A | CSK | 1445 | | | 16139795 | | | |
| NS5A | DNAJA3 | 9093 | | 1 | | | | |
| NS5A | EFEMP1 | 2202 | | 2 | | | + | |
| NS5A | EIF2AK2 | 5610 | | | 9143277\| | | | |
| | | | | | 9710605\| | | | |
| | | | | | 10488152\| | | | |
| | | | | | 12634350 | | | |
| NS5A | FBL2 | 25827 | | | 15893726 | | | |
| NS5A | FBXL2 | 25827 | | | | 15576676\| | | |
| | | | | | | 15893726 | | |
| NS5A | FHL2 | 2274 | | 1 | | | NA | |
| NS5A | FTH1 | 2495 | | | 15607035 | 15607035 | | |
| NS5A | FYN | 2534 | | | 15784897 | 14993658 | | |
| NS5A | GOLGA2 | 2801 | | 15 | | | + | |
| NS5A | GPS2 | 2874 | | 1 | | | + | |
| NS5A | GRB2 | 2885 | | | 10318918\| | 10318918 | | |
| | | | | | 12186904\| | | | |
| | | | | | 12556990\| | | | |
| | | | | | 15784897 | | | |
| NS5A | GSK3A | 2931 | | | | 17616579 | | |
| NS5A | GSK3B | 2931 | | | | 17616579 | | |
| NS5A | HCK | 3055 | | | 15784897 | 14993658 | | |
| NS5A | IGLL1 | 3537 | | 1 | | | | |
| NS5A | IPO4 | 79711 | | | | 17616579 | | |
| NS5A | ITGAL | 3683 | | 1 | | | + | |
| NS5A | JAK1 | 3716 | | | | 15063116 | | |
| NS5A | LCK | 3932 | | | 15784897 | 14993658 | | |
| NS5A | LIMS2 | 55679 | | 1 | | | | |
| NS5A | LOC374395 | 374395 | | 1 | | | | |
| NS5A | LYN | 4067 | | | 15784897 | 14993658 | | |
| NS5A | MAPK12 | 6300 | | | | 17616579 | | |
| NS5A | MGC2574 | 79080 | | | | 15607035 | | |
| NS5A | MGP | 4155 | | | 15607035 | 15607035 | | |
| NS5A | MOBK1B | 55233 | | 5 | | | + | |
| NS5A | NAP1L1 | 4673 | 1 | 31 | | | NA | + |
| NS5A | NAP1L2 | 4674 | | 4 | | | + | |
| NS5A | NDRG1 | 10397 | | | 15607035 | 15607035 | | |
| NS5A | NFE2 | 4778 | | 1 | | | NA | |
| NS5A | NUCB1 | 4924 | | 2 | | | NA | |
| NS5A | OAS1 | 4938 | | | 15039538 | | | |
| NS5A | PARVG | 64098 | | 1 | | | NA | |
| NS5A | PDPK1 | 5170 | | | | 17616579 | | |
| NS5A | PIK3R1 | 5291 | | | 14709551 | | | |
| NS5A | PIK4CA | 5297 | | | 15607035 | 15607035 | | |
| NS5A | PITX1 | 5307 | | | 12620797 | | | |
| NS5A | PMVK | 10654 | | 1 | | | NA | |
| NS5A | PPP1R13L | 10848 | | 2 | | | + | |
| NS5A | PSMB9 | 5698 | | 38 | | | + | |
| NS5A | PTMA | 5757 | | | 15607035 | 15607035 | | |
| NS5A | RAF1 | 5894 | | | 16405965 | 17616579 | | |
| NS5A | RANBP5 | 3843 | | | 10799599 | 10799599 | | |
| NS5A | RPL18A | 6142 | | 1 | | | | |
| NS5A | RRBP1 | 6238 | | 2 | | | | |
| NS5A | SFRP4 | 6424 | | | 15607035 | 15607035 | | |
| NS5A | SHARPIN | 81858 | | 2 | | | | |
| NS5A | SMYD3 | 64754 | 1 | | | | | + |
| NS5A | SORBS2 | 8470 | | 8 | | | + | |
| NS5A | SORBS3 | 10174 | | 1 | | | | |
| NS5A | SRC | 6714 | | | 16139795 | | | |
| NS5A | SRCAP | 10847 | | | 10702287 | 10702287 | | |
| NS5A | SSB | 6741 | | | | 12963047 | | |
| NS5A | TACSTD2 | 4070 | | | 15607035 | 15607035 | | |
| NS5A | TAF9 | 6880 | | | 12101418 | 12101418 | | |
| NS5A | TBP | 6908 | | | 12379483 | 12379483\| | | |
| | | | | | | 7862623\| | | |
| | | | | | | 9143277 | | |
| NS5A | THBS1 | 7057 | | 7 | | | | |
| NS5A | TMF1 | 7110 | | 2 | | | − | |
| NS5A | TP53 | 7157 | | | 12101418\| | 12379483\| | | |
| | | | | | 12379483 | 11152513\| | | |
| | | | | | | 12101418 | | |
| NS5A | TP53BP2 | 7159 | | 2 | | | | |
| NS5A | TRAF2 | 7186 | | | 11821416 | 11821416 | | |
| NS5A | TRIOBP | 11078 | | 2 | | | NA | |
| NS5A | TXNDC11 | 51061 | | 1 | | | + | |
| NS5A | UBASH3A | 53347 | | 1 | | | | |
| NS5A | USP19 | 10869 | 3 | | | | | + |
| NS5A | VAPA | 9218 | | | 10544080\| | 10544080\| | | |
| | | | | | 15016871\| | 15016871\| | | |
| | | | | | 15326295\| | 15326295 | | |
| | | | | | 16227268 | | | |
| NS5A | VAPB | 9217 | | | 16227268 | | | |
| NS5A | VPS35 | 55737 | | | | 17616579 | | |
| NS5A | VPS52 | 6293 | | 2 | | | + | |
| NS5A | ZH2C2 | 54826 | | 2 | | | | |
| NS5A | ZNF646 | 9726 | | 2 | | | − | |
| NS5B | ACTN1 | 87 | | | | 14623081 | | |
| NS5B | CEP250 | 11190 | | 1 | | | | |
| NS5B | CEP68 | 23177 | 1 | | | | | + |
| NS5B | CHUK | 1147 | | | 16581780 | | | |
| NS5B | DDX5 | 1655 | | | | 11556407\| | | |
| | | | | | | 15113910 | | |
| NS5B | EIF4A2 | 1974 | | | 11922617 | 11922617 | | |
| NS5B | FBXL2 | 25827 | | | | 15893726 | | |
| NS5B | HAO1 | 54363 | | | | 14623081 | | |
| NS5B | HOXD8 | 3234 | 1 | | | | | + |
| NS5B | MGC2752 | 65996 | 1 | | | | | + |
| NS5B | MOBK1B | 55233 | | 3 | | | NA | |
| NS5B | NCL | 4691 | | | 12427757\| | 12427757 | | |
| | | | | | 16537600 | | | |
| NS5B | NR4A1 | 3164 | 8 | | | | | + |
| NS5B | OS9 | 10956 | | 3 | | | NA | |
| NS5B | PKM2 | 5315 | | 2 | | | | |
| NS5B | PKN2 | 5586 | | | 15364941 | | | |
| NS5B | PPIB | 5479 | | | 15989969 | 15989969 | | |
| NS5B | PSMB9 | 5698 | | 14 | | | NA | |
| NS5B | PTBP2 | 58155 | | | 15823607 | | | |
| NS5B | SETD2 | 29072 | 3 | | | | | + |
| NS5B | SHARPIN | 81858 | | 1 | | | | |
| NS5B | TTC4 | 7268 | | | | 14623081 | | |
| NS5B | TUBB2C | 10383 | 1 | | | | | + |
| NS5B | UBQLN1 | 29979 | | | | 12634373 | | |
| NS5B | VAPA | 9218 | | | 10544080\| | 10544080\| | | |
| | | | | | 15016871\| | 15016871 | | |
| | | | | | 16227268 | | | |
| NS5B | VAPB | 9217 | | | 16227268 | | | |
| p7 | CREB3 | 10488 | | 1 | | | NA | |
| p7 | FBLN2 | 2199 | 1 | | | | | + |
| p7 | FMNL1 | 752 | | | 16094715 | | | |
| p7 | FXYD6 | 53826 | 1 | | | | | + |
| p7 | H19 | 283120 | | | 16094715 | | | |
| p7 | ISLR | 3671 | | | 16094715 | | | |
| p7 | LMNB1 | 4001 | 1 | | | | | + |
| p7 | MS4A6A | 64231 | | | 16094715 | | | |
| p7 | NUP214 | 8021 | | | 16094715 | | | |
| p7 | SLIT2 | 9353 | 1 | | | | | + |
| p7 | SSR4 | 6748 | | | 16094715 | | | |
| p7 | STRBP | 55342 | | | 16094715 | | | |
| p7 | UBQLN1 | 29979 | 3 | | | | | + |
| p7 | UBQLN4 | 56893 | 3 | | | | | + |

TABLE S2

| Human | | |
|---|---|---|
| protein | HCV protein | Interaction datasets |
| ACTN1 | NS3, NS5B | Y2H, LCI |
| CALR | E1, E2 | LCI, LCI |
| CANX | E1, E2 | LCI, LCI |
| CD209 | E1, E2 | LCI, LCI |
| CEP250 | NS5A, NS5B | Y2H, Y2H |
| CLEC4M | E1, E2 | LCI, LCI |
| CREB3 | NS4A, p7 | Y2H, Y2H |
| DDX5 | CORE, NS5B | LCI, LCI |
| EFEMP1 | NS3, NS5A | Y2H, Y2H |
| EIF2AK2 | E2, NS5A | LCI, LCI |
| FBLN2 | CORE, NS3, p7 | Y2H, Y2H, Y2H |
| FBLN5 | CORE, NS2, NS3 | Y2H, Y2H, Y2H |
| FBXL2 | NS5A, NS5B | LCI, LCI |
| FTH1 | NS3, NS5A | Y2H, LCI |
| HIVEP2 | CORE, NS3 | Y2H, Y2H |
| HNRPK | CORE, NS3 | LCI, Y2H |
| HOXD8 | CORE, E2, NS2, | Y2H, Y2H, Y2H, |
| | NS4A, NS5B | Y2H, Y2H |
| HSPA5 | E1, E2 | LCI, LCI |
| JAK1 | CORE, NS5A | LCI, LCI |
| JUN | E1, NS3 | Y2H, Y2H |
| LTBP4 | CORE, NS3 | Y2H, Y2H |
| LTF | E1, E2 | LCI, LCI |
| MAGED1 | CORE, NS3 | Y2H, Y2H |
| MOBK1B | NS5A, NS5B | Y2H, Y2H |
| NAP1L1 | NS3, NS5A | Y2H; Y2H |
| NR4A1 | CORE, E1, E2, | Y2H, Y2H, Y2H, |
| | NS2, NS4A, NS5B | Y2H, Y2H, Y2H |
| PSMB9 | NS3, NS5A, NS5B | Y2H, Y2H, Y2H |
| PSME3 | CORE, NS3 | LCI, Y2H |
| PTBP2 | NS3, NS5B | LCI, LCI |
| RNF31 | CORE, NS3 | Y2H, Y2H |
| SETD2 | CORE, E1, E2, | Y2H, Y2H, Y2H, |
| | NS2, NS5B | Y2H, Y2H |
| SHARPIN | NS5A, NS5B | Y2H, Y2H |
| SLIT2 | NS3, p7 | Y2H, Y2H |
| SMAD3 | CORE, NS3 | LCI, LCI |
| STAT3 | CORE, NS3 | LCI, Y2H |
| TBP | CORE, NS5A | LCI, LCI |
| TP53 | CORE, NS3, NS5A | LCI, LCI, LCI |
| TP53BP2 | CORE, NS5A | LCI, Y2H |
| TRIM27 | NS2, NS3 | Y2H, Y2H |
| TXNDC11 | NS3, NS5A | Y2H, Y2H |
| UBQLN1 | NS4A, NS5B, p7 | Y2H, LCI, Y2H |
| VAPA | NS5A, NS5B | LCI, LCI |
| VAPB | NS5A, NS5B | LCI, LCI |
| VIM | CORE, NS3 | LCI, Y2H |
| VWF | CORE, NS3 | Y2H, Y2H |

TABLE S3
Connected Components of Virus-Human network
Sim = Random Network simulation (1000 replicates)

| | IMAP dataset | IMAP Sim |
|---|---|---|
| maximum size of | 196(***) | 17.9 |
| connected components | | |
| (comparison to | | |
| simulation*** p- | | |
| value <10e−10) | | |
| number of connected | 131(***) | 276.2 |
| components | | |
| (comparison to | | |
| simulation*** p- | | |
| value <10e−10) | | |

Topological Analysis of Virus-Human network

| | | | | | |
|---|---|---|---|---|---|
| DATASET | proteins | ppi | k | b (10e−4) | l |

A. Integration of H_{HCV} and H_{EBV} with Human “full” protein interactome.

| H in H-H network Full | | 9520 | 44223 | 9.3 | 1.6 | 4.04 |
|---|---|---|---|---|---|---|
| H_{HCV} in H-H network Full | | | | | | |
| IMAP Y2H | Y2H | 213 (76%) | 72 | 15.6 | 3.8 | 3.50 |
| IMAP LCI | TEXT-MINING | 107 (87%) | 142 | 43.2 | 14.4 | 3.00 |
| | BIND | 64 (94%) | 60 | 54.8 | 20.0 | 2.98 |
| | LCI_TOTAL | 135 (88%) | 221 | 43.4 | 14.6 | 2.99 |
| IMAP | TOTAL | 338 (80%) | 445 | 25.6 | 7.7 | 3.36 |

| | | | | | |
|---|---|---|---|---|---|
| H_{EBV} in H-H network Full | 91 (80%) | 21 | 23 | 6.5 | 3.23 |

B. Integration of H_{HCV} and H_{EBV} with Human high confidence protein interactome.

| H in H-H network high confidence | | 5883 | 16877 | 5.7 | 3.2 | 4.8 |
|---|---|---|---|---|---|---|
| H_{HCV} in H-H network high confidence | | | | | | |
| IMAP Y2H | Y2H | 154 (55%) | 22 | 7.2 | 4.7 | 4.37 |
| IMAP LCI | TEXT-MINING | 94 (77%) | 76 | 23 | 25.4 | 3.72 |
| | BIND | 58 (85%) | 32 | 28.1 | 32 | 3.76 |
| | LCI TOTAL | 120 (78%) | 125 | 23.5 | 26 | 3.67 |
| IMAP | TOTAL | 264 (62%) | 222 | 14.1 | 14 | 4.12 |

| | | | | | |
|---|---|---|---|---|---|
| H_{EBV} in H-H network high confidence | 71 (62%) | 5 | 8.4 | 6.0 | 4.28 |

TABLE S4

| kegg_name | CORE | E1 | E2 | NS3 | NS5A | NS5B |
|---|---|---|---|---|---|---|
| Cell-cell and cell-ECM interactions | | | | | | |
| Adherens junction | 5 | | | 6(5) | | |
| Cell communication | 6(2) | | | 8(8) | | |
| Cell adhesion molecules (CAMs) | | | 3(1) | | | |
| ECM-receptor interaction | | | 2(1) | 6(6) | | |
| Focal adhesion | | | | 10(9) | 8(2) | |
| Gap junction | | | | | 4 | |
| Tight junction | | | | | | 2 |
| Signaling pathways | | | | | | |
| TGF-beta signaling pathway | 4 | | | | | |
| Jak-STAT signaling pathway | 6 | | | | 3 | |
| Adipocytokine signaling pathway | 5 | | | | | |
| MAPK signaling pathway | | 3(2) | | | | |
| Phosphatidylinositol signaling system | | | 2 | | 4 | |
| Wnt signaling pathway | | | | 7(3) | | |
| Insulin signaling pathway | | | | | 3 | |
| B cell receptor receptor signaling | | | | | 3 | |
| pathway | | | | | | |
| T cell receptor signaling pathway | | | | | 5 | |
| Toll-like receptor signaling pathway | | | | | | |

TABLE S5
A. HHCV enrichment in IJT network for each viral protein

| | | | | | p-value |
|---|---|---|---|---|---|
| | H_{HCV} in V- | H_{HCV} in IJT | H_{HCV} in | | (Fisher |
| | H_{HCV} | network | IJT (%) | enrichment | test) |
| NS3 | 214 | 38 | 17.8 | 0.33 | 1.000 |
| NS5A | 96 | 32 | 33.3 | 1.23 | 0.229 |
| CORE | 76 | 39 | 51.3 | 3.04 | 1.30E−05 |
| NS5B | 26 | 11 | 42.3 | 1.79 | 0.113 |
| E2 | 20 | 8 | 40.0 | 1.61 | 0.215 |
| P7 | 14 | 2 | 14.3 | 0.39 | 0.953 |
| E1 | 11 | 6 | 54.5 | 2.91 | 0.073 |
| F | 9 | 2 | 22.2 | 0.67 | 0.802 |
| NS2 | 8 | 2 | 25.0 | 0.79 | 0.742 |
| NS4A | 6 | 3 | 50.0 | 2.39 | 0.250 |
| NS4B | 1 | 0 | 0.0 | 0.00 | 1.000 |

B. H_{HCV} enrichment in main pathways for each viral protein

| | | | Jak/ | | | | TGFb p- | | | Insulin p- |
|---|---|---|---|---|---|---|---|---|---|---|
| | H_{HCV} | | STAT | Jak/STAT p- | | TGFb | value | | Insulin | value |
| | in V- | Jak/ | enrich- | value | | enrich- | (Fisher | | enrich- | (Fisher |
| | H_{HCV} | Stat | ment | (Fisher test) | TGFb | ment | test) | Insulin | ment | test) |
| NS3 | 214 | 1 | 0.13 | 9.97E−01 | 2 | 0.50 | 8.94E−01 | 1 | 0.3 | 9.48E−01 |
| NS5A | 96 | 3 | 1.74 | 3.20E−01 | 1 | 0.67 | 7.92E−01 | 3 | 6.1 | 5.66E−02 |
| CORE | 76 | 6 | 8.53 | 1.61E−03 | 4 | 7.39 | 1.39E−02 | 0 | 0.0 | 1.00E+00 |
| NS5B | 26 | 0 | 0.00 | 1.00E+00 | 0 | 0.00 | 1.00E+00 | 1 | 4.5 | 2.43E−01 |
| E2 | 20 | 0 | 0.00 | 1.00E+00 | 0 | 0.00 | 1.00E+00 | 0 | 0.0 | 1.00E+00 |
| P7 | 14 | 0 | 0.00 | 1.00E+00 | 0 | 0.00 | 1.00E+00 | 0 | 0.0 | 1.00E+00 |
| E1 | 11 | 0 | 0.00 | 1.00E+00 | 0 | 0.00 | 1.00E+00 | 0 | 0.0 | 1.00E+00 |
| F | 9 | 0 | 0.00 | 1.00E+00 | 0 | 0.00 | 1.00E+00 | 0 | 0.0 | 1.00E+00 |
| NS2 | 8 | 0 | 0.00 | 1.00E+00 | 0 | 0.00 | 1.00E+00 | 0 | 0.0 | 1.00E+00 |
| NS4A | 6 | 0 | 0.00 | 1.00E+00 | 0 | 0.00 | 1.00E+00 | 0 | 0.0 | 1.00E+00 |
| NS4B | 1 | 0 | 0.00 | 1.00E+00 | 0 | 0.00 | 1.00E+00 | 0 | 0.0 | 1.00E+00 |

C. H_{HCV} enrichment in interpathways for each viral protein

| | | | | | | | Jak/STAT- |
|---|---|---|---|---|---|---|---|
| | | | | Insulin- | | | TGFb |
| | | | Insulin- | Jak/Stat p- | Jak/STAT- | | p-value |
| | H_{HCV} in V- | Insulin- | Jak/Stat | value | TGFb | Jak/STAT- | (Fisher |
| | H_{HCV} | Jak/Stat | enrichment | (Fisher test) | enrichment | TGFb | test) |
| NS3 | 214 | 23 | 0.44 | 9.99E−01 | 15 | 0.37 | 1.00E+00 |
| NS5A | 96 | 15 | 0.91 | 6.67E−01 | 17 | 1.71 | 6.27E−02 |
| CORE | 76 | 22 | 2.43 | 2.35E−03 | 13 | 1.57 | 1.28E−01 |
| NS5B | 26 | 8 | 2.36 | 4.99E−02 | 6 | 2.22 | 9.05E−02 |
| E2 | 20 | 5 | 1.71 | 2.26E−01 | 2 | 0.77 | 7.38E−01 |
| P7 | 14 | 0 | 0.00 | 1.00E+00 | 1 | 0.53 | 8.49E−01 |
| E1 | 11 | 5 | 4.37 | 2.29E−02 | 2 | 1.58 | 4.08E−01 |
| F | 9 | 0 | 0.00 | 1.00E+00 | 0 | 0.00 | 1.00E+00 |
| NS2 | 8 | 1 | 0.71 | 7.69E−01 | 2 | 2.38 | 2.62E−01 |
| NS4A | 6 | 1 | 1.00 | 6.66E−01 | 2 | 3.58 | 1.65E−01 |
| NS4B | 1 | 0 | 0.00 | 1.00E+00 | 0 | 0.00 | 1.00E+00 |

| | | TGFb- | | | | Insulin- |
|---|---|---|---|---|---|---|
| | | Insulin | TGFb- | | Insulin- | Jak/STAT- |
| | TGFb- | p-value | Insulin | Insulin- | Jak/STAT- | TGFb p- |
| | Insulin | (Fisher | (Fisher | Jak/STAT- | TGFb | value (Fisher |
| | enrichment | test) | test) | TGFb | enrichment | test) |
| NS3 | 16 | 0.40 | 9.99E−01 | 10 | 0.39 | 9.98E−01 |
| NS5A | 11 | 0.87 | 7.11E−01 | 9 | 1.18 | 4.03E−01 |
| CORE | 16 | 2.13 | 1.75E−02 | 11 | 2.19 | 3.51E−02 |
| NS5B | 6 | 2.18 | 9.66E−02 | 5 | 2.85 | 5.48E−02 |
| E2 | 3 | 1.23 | 4.77E−01 | 1 | 0.57 | 8.30E−01 |
| P7 | 1 | 0.52 | 8.54E−01 | 0 | 0.00 | 1.00E+00 |
| E1 | 3 | 2.66 | 1.52E−01 | 2 | 2.52 | 2.31E−01 |
| F | 2 | 2.00 | 3.19E−01 | 0 | 0.00 | 1.00E+00 |
| NS2 | 1 | 0.98 | 6.65E−01 | 1 | 1.59 | 5.03E−01 |
| NS4A | 2 | 3.51 | 1.70E−01 | 1 | 2.23 | 4.08E−01 |
| NS4B | 0 | 0.00 | 1.00E+00 | 0 | 0.00 | 1.00E+00 |

1. A method for screening compounds for treating and/or preventing an HCV infection comprising the steps of:
a) determining the ability of a candidate compound to inhibit the interaction between the farnesoid X receptor (FXR) and viral HCV protein NS3 or NS5A;
b) selecting the candidate compound that inhibits said interaction between said viral farnesoid X receptor (FXR) and said viral protein.

2. A method for screening compounds for treating and/or preventing an HCV infection comprising the steps of:
a) determining the ability of a candidate compound to inhibit the interaction between the viral HCV CORE protein and a human protein selected from the group consisting of AGRN, BCAR1, CD68, COL4A2, DDX3Y, EGFL7, FBLN2, FBLN5, GAPDH, GRN, HIVEP2, HOXD8, LPXN, LRRTM1, LTBP4, MAGED1, MEGF6, MMRN2, NR4A1, PABPN1, PAK4, PLSCR1, RNF31, SETD2, SLC31A2, VTN, VWF, and ZNF271; and
b) selecting the candidate compound that inhibits said interaction between said viral HCV CORE protein and said human protein.

3. A method for screening compounds for treating and/or preventing an HCV infection comprising the steps of:
a) determining the ability of a candidate compound to inhibit the interaction between the viral HCV E1 protein and a human protein selected from the group consisting of JUN, NR4A1, PFN1, SETD2, and TMSB4X; and
b) selecting the candidate compound that inhibits said interaction between said viral E1 protein and said human protein.

4. A method for screening compounds for treating and/or preventing an HCV infection comprising the steps of:
a) determining the ability of a candidate compound to inhibit the interaction between the viral HCV E2 protein and a human protein selected from the group consisting of HOXD8, ITGB1, KIAA1411, LOC730765, NR4A1, PSMA6, SETD2, and SMEK2; and
b) selecting the candidate compound that inhibits said interaction between said viral E2 protein and said human protein.

5. A method for screening compounds for treating and/or preventing an HCV infection comprising the steps of:
a) determining the ability of a candidate compound to inhibit the interaction between the viral HCV NS2 protein and a human protein selected from the group consisting of ADFP, APOA1, C7, FBLN5, HOXD8, NR4A1, POU3F2, RPL11, RPN1, SETD2, SMURF2, and TRIM27; and
b) selecting the candidate compound that inhibits said interaction between said viral NS2 protein and said human protein.

6. A method for screening compounds for treating and/or preventing an HCV infection comprising the steps of:
a) determining the ability of a candidate compound to inhibit the interaction between the viral HCV NS3 protein and a human protein selected from the group consisting of sep-10, A1BG, ABCC3, ACTN1, ACTN2, AEBP1, AHCY, AHSG, ALB, ANKRD12, ANKRD28, APOA1, APOA2, ARFIP2, ARG1, ARHGDIA, ARHGEF6, ARNT, ARS2, ASXL1, ATP5H, AZGP1, B2M, BCAN, BCKDK, BCL2A1, BCL6, BCR, BZRAP1, C10orf18, C10orf6, C12orf41, C14orf173, C16orf7, C1orf165, C1orf94, C1S, C9orf30, CALCOCO2, CAT, CBY1, CCDC21, CCDC37, CCDC52, CCDC66, CCDC95, CCHCR1, CCNDBP1, CD5L, CDC23, CELSR2, CENPC1, CEP152, CEP192, CES1, CFP, CHPF, COL3A1, CORO1B, COX3, CSNK2B, CTGF, CTSD, CTSF, CXorf45, DEAF1, DEDD2, DES, DLAT, DOCK7, DPF1, DPP7, ECHS1, EEF1A1, EFEMP1, EFEMP2, EIF1, EIF4ENIF1, FAM120B, FAM62B, FAM65A, FAM96B, FBF1, FBLN1, FBLN2, FBLN5, FBN1, FBN3, FES, FGA, FGB, FIGNL1, FLAD1, FLJ11286, FN1, FRMPD4, FRS3, FTH1, FUCA2, GAA, GBP2, GC, GFAP, GNB2, GON4L, HIVEP2, HOMER3, HP, HTRA1, IFI44, IQWD1, ITCH, ITGB4, JAG2, JUN, KHDRBS1, KIAA1012, KIAA1549, KIF17, KIF7, KNG1, KPNA1, KPNB1, L3 MBTL3, LAMA5, LAMB2, LAMC3, LDB1, LOC728302, LRRC7, LRRCC1, LTBP4, LZTS2, MAGED1, MAPK7, MARCO, MASP2, MEGF8, MLLT4, MLXIP, MORC4, MORF4L1, MPDZ, MVP, MYL6, NAP1L1, NCAN, NDC80, NEFL, NEFM, NELL1, NELL2, NID1, NID2, NOTCH1, N-PAC, NUCB1, NUP133, NUP62, OBSCN, ORM1, OTC, PARP2, PARP4, PCYT2, PDE4DIP, PDLIM5, PGM1, PICK1, PKNOX1, PLEKHG4, PNPLA8, PNPT1, POLDIP2, PRG4, PRRC1, PSMA6, PSMB9, PSME3, PTPRF, PTPRN2, RABEP1, RAI14, RASAL2, RBM4, RCN3, RGNEF, RICS, RING1, RINT1, RLF, RNF31, ROGDI, RP11-130N24.1, RSHL2, RUSC2, SBF1, SDCCAG8, SECISBP2, SELO, SERTAD1, SESTD1, SF3B2, SGCB, SIAH1, SLIT1, SLIT2, SLIT3, SMARCE1, SMURF2, SNX4, SPOCK3, SPON1, SPP2, SRPX2, SSX21P, STAB1, STAT3, STRAD, SVEP1, SYNE1, SYNPO2, TAF1, TAF15, TBC1D2B, TBN, TBXAS1, TF, TGFB1I1, TH1L, THAP1, TMEM63B, TPST2, TPT1, TRIM23, TRIM27, TRIO, TRIP11, TXNDC11, UBE1C, USHBP1, UXT, VCAN, VIM, VWF, WDTC1, XAB2, XRN2, YY1AP1, ZADH1, ZBTB1, ZCCHC7, ZHX3, ZMYM2, ZNF281, ZNF410, ZNF440, ZXDC, and ZZZ3, APOA1, and DNAJB1; and
b) selecting the candidate compound that inhibits said interaction between said viral NS3 protein and said human protein.

7. A method for screening compounds for treating and/or preventing an HCV infection comprising the steps of:
a) determining the ability of a candidate compound to inhibit the interaction between the viral HCV NS4A protein and a human protein selected from the group consisting of CREB3, ELAC2, HOXD8, NR4A1, TRAF3IP3, UBQLN1, APOA1, and DNAJB1; and
b) selecting the candidate compound that inhibits said interaction between said viral NS4A protein and said human protein.

8. A method for screening compounds for treating and/or preventing an HCV infection comprising the steps of:
a) determining the ability of a candidate compound to inhibit the interaction between the viral HCV NS4B protein and a human protein selected from the group consisting of APOA1, ATF6, KNG1, and NR4A1; and
b) selecting the candidate compound that inhibits said interaction between said viral NS4B protein and said human protein.

9. A method for screening compounds for treating and/or preventing an HCV infection comprising the steps of:
a) determining the ability of a candidate compound to inhibit the interaction between the viral HCV NS5A protein and a human protein selected from the group consisting of AARS2, ABCC3, ACLY, ACTB, ALDOB, APOB, ARFIP1, ASXL1, AXIN1, C10orf30, C9orf23, CADPS, CADPS2, CCDC100, CCDC90A, CCT7, CEP250, CEP63, CES1, CFH, COL3A1, DDX5, DNAJA3, EFEMP1, EIF3S2, ETFA, FGB, FHL2, GLTSCR2, GOLGA2, GPS2, HRSP12, IGLL1, ITGAL, LDHD, LIMS2, LOC374395, MAF, MBD4, MKRN2, MOBK1B, MON2, NAP1L1, NFE2, NUCB1, OS9, PARVG, PMVK, POMP, PPP1R13L, PSMB8, PSMB9, RLF, RPL18A, RRBP1, SHARPIN, SMYD3, SORBS2, SORBS3, THBS1, TMF1, TP53BP2, TRIOBP, TST, TXNDC11, UBASH3A, UBC, USP19, VPS52, ZGPAT, ZH2C2, ZNF135, ZNF350, ZNF646, ZNHIT1, and ZNHIT4; and
b) selecting the candidate compound that inhibits said interaction between said viral NS5A protein and said human protein.

10. A method for screening compounds for treating and/or preventing an HCV infection comprising the steps of:
a) determining the ability of a candidate compound to inhibit the interaction between the viral HCV NS5B protein and a human protein selected from the group consisting of APOA1, APOC3, CCNDBP1, CEP250, CEP68, CTSF, HOXD8, MGC2752, MOBK1B, OS9, OTC, PKM2, PSMB9, SETD2, SHARPIN, TAGLN and TUBB2C; and
b) selecting the candidate compound that inhibits said interaction between said viral NS5B protein and said human protein.

11. A method for screening compounds for treating and/or preventing an HCV infection comprising the steps of:
a) determining the ability of a candidate compound to inhibit the interaction between the viral HCV p7 protein and a human protein selected from the group consisting of CREB3, FBLN2, FXYD6, LMNB1, RNUXA, SLC39A8, SLIT2, UBQLN1, and UBQLN4; and
b) selecting the candidate compound that inhibits said interaction between said viral HCV p7 protein and said human protein.
